(19) United States
(12) Patent Application Publication
(10) Pub. No.: US 2009/0175852 A1
(43) Pub. Date: Jul. 9, 2009

(54) IMIDAZOPYRAZINES AS PROTEIN KINASE INHIBITORS

(75) Inventors: Jeffrey P. Ciavarri, Reading, MA (US); Panduranga Adulla P. Reddy, Walpole, MA (US); M. Arshad Siddiqui, Newton, MA (US); Lianyun Zhao, Burlington, MA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/362,367

(22) Filed: Jan. 29, 2009

Related U.S. Application Data
(63) Continuation-in-part of application No. 11/758,243, filed on Jun. 5, 2007.
(60) Provisional application No. 60/811,351, filed on Jun. 6, 2006.

(51) Int. Cl. A61K 31/4985; C07D 487/04; A61K 39/395; A61P 35/00

(52) U.S. Cl. 424/130.1; 544/350; 514/250

(57) ABSTRACT

In its many embodiments, the present invention provides a novel class of imidazopyrazine compounds as inhibitors of protein and/or checkpoint kinases, methods of preparing such compounds, pharmaceutical compositions including one or more such compounds, methods of preparing pharmaceutical formulations including one or more such compounds, and methods of treatment, prevention, inhibition, or amelioration of one or more diseases associated with the protein or checkpoint kinases using such compounds or pharmaceutical compositions.

### REFERENCE TO RELATED APPLICATIONS

This application is a Continuation-in-Part of U.S. patent application Ser. No. 11/758,243, filed Jun. 5, 2007 (which published as US 2008/0027063 on Jan. 31, 2008), which claims the benefit of U.S. provisional application Ser. No. 60/811,351 filed Jun. 6, 2006.

### FIELD OF THE INVENTION

The present invention relates to imidazo[1,2-a]pyrazine compounds useful as protein kinase inhibitors, regulators or modulators, pharmaceutical compositions containing the compounds, and methods of treatment using the compounds and compositions to treat diseases such as, for example, cancer, inflammation, arthritis, viral diseases, neurodegenerative diseases such as Alzheimer's disease, cardiovascular diseases, and fungal diseases.

### BACKGROUND OF THE INVENTION

Protein kinases are a family of enzymes that catalyze phosphorylation of proteins, in particular the hydroxyl group of specific tyrosine, serine, or threonine residues in proteins. Protein kinases are pivotal in the regulation of a wide variety of cellular processes, including metabolism, cell proliferation, cell differentiation, and cell survival. Uncontrolled proliferation is a hallmark of cancer cells, and can be manifested by a deregulation of the cell division cycle in one of two ways—making stimulatory genes hyperactive or inhibitory genes inactive. Protein kinase inhibitors, regulators or modulators alter the function of kinases such as cyclin-dependent kinases (CDKs), mitogen activated protein kinase (MAPK/ERK), glycogen synthase kinase 3 (GSK3beta), Checkpoint (Chk) (e.g., CHK-1, CHK-2 etc.) kinases, AKT kinases, JNK, and the like. Examples of protein kinase inhibitors are described in WO02/22610 A1 and by Y. Mettey et al in J. Med. Chem., (2003) 46 222-236.

The cyclin-dependent kinases are serine/threonine protein kinases, which are the driving force behind the cell cycle and cell proliferation. Misregulation of CDK function occurs with high frequency in many important solid tumors, Individual CDK's, such as, CDK1, CDK2, CDK3, CDK4, CDK5, CDK6 and CDK7, CDK8 and the like, perform distinct roles in cell cycle progression and can be classified as either G1, S, or G2M phase enzymes. CDK2 and CDK4 are of particular interest because their activities are frequently misregulated in a wide variety of human cancers. CDK2 activity is required for progression through G1 to the S phase of the cell cycle, and CDK2 is one of the key components of the G1 checkpoint. Checkpoints serve to maintain the proper sequence of cell cycle events and allow the cell to respond to insults or to proliferative signals, while the loss of proper checkpoint control in cancer cells contributes to tumorgenesis. The CDK2 pathway influences tumorgenesis at the level of tumor suppressor function (e.g. p52, RB, and p27) and oncogene activation (cyclin E). Many reports have demonstrated that both the coactivator, cyclin E, and the inhibitor, p27, of CDK2 are either over- or underexpressed, respectively, in breast, colon, nonsmall cell lung, gastric, prostate, bladder, non-Hodgkin's lymphoma, ovarian, and other cancers. Their altered expression has been shown to correlate with increased CDK2 activity levels and poor overall survival. This observation makes CDK2 and its regulatory pathways compelling targets for the development of cancer treatments.

A number of adenosine 5′-triphosphate (ATP) competitive small organic molecules as well as peptides have been reported in the literature as CDK inhibitors for the potential treatment of cancers. U.S. Pat. No. 6,413,974, col. 1, line 23-col. 15, line 10 offers a good description of the various CDKs and their relationship to various types of cancer. Flavopiridol (shown below) is a nonselective CDK inhibitor that is currently undergoing human clinical trials, A. M. Sanderowicz et al., J. Clin. Oncol. (1998) 16, 2986-2999.

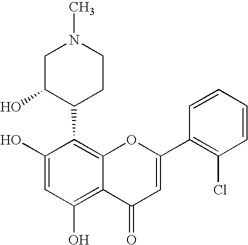

Other known inhibitors of CDKs include, for example, olomoucine (J. Vesely et al, Eur. J. Biochem., (1994) 224, 771-786) and roscovitine (I. Meijer et al, Eur. J. Biochem., (1997) 243, 527-536). U.S. Pat. No. 6,107,305 describes certain pyrazolo[3,4-b]pyridine compounds as CDK inhibitors. An illustrative compound from the '305 patent is:

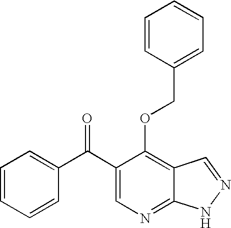

K. S. Kim et al, J. Med. Chem. 45 (2002) 3905-3927 and WO 02/10162 disclose certain aminothiazole compounds as CDK inhibitors.

Imidazopyrazines are known. For example, U.S. Pat. No. 6,919,341 (the disclosure of which is incorporated herein by reference) and US2005/0009832 disclose various imidazopyrazines. Also being mentioned are the following: WO2005/047290; US2005/095616; WO2005/039393; WO2005/019220; WO2004/072081; WO2005/014599; WO2005/009354; WO2005/005429; WO2005/085252; US2005/009832; US2004/220189; WO2004/074289; WO2004/026877; WO2004/026310; WO2004/022562; WO2003/089434; WO2003/084959; WO2003/051346; US2003/022898; WO2002/060492; WO2002/060386; WO2002/028860; JP (1986)61-057587; J. Burke et al. J. Biological Chem., Vol. 278(3), 1450-1456 (2003); and F. Bondavalli et al, J. Med. Chem., Vol. 45 (22), 4875-4887 (2002). Additionally, commonly owned, U.S. Pat. No. 6,919,341, and pending U.S. provisional patent application Ser. No. 60/735,982 filed Nov. 10, 2005, and incorporated herein by reference, disclose several imidazo[1,2-a]pyrazines.

Another series of protein kinases are those that play an important role as a checkpoint in cell cycle progression. Checkpoints prevent cell cycle progression at inappropriate times, such as in response to DNA damage, and maintain the metabolic balance of cells while the cell is arrested, and in some instances can induce apoptosis (programmed cell death) when the requirements of the checkpoint have not been met. Checkpoint control can occur in the G1 phase (prior to DNA synthesis) and in G2, prior to entry into mitosis.

One series of checkpoints monitors the integrity of the genome and, upon sensing DNA damage, these “DNA damage checkpoints” block cell cycle progression in G₁ & G₂ phases, and slow progression through S phase. This action enables DNA repair processes to complete their tasks before replication of the genome and subsequent separation of this genetic material into new daughter cells takes place. Inactivation of CHK1 has been shown to transduce signals from the DNA-damage sensory complex to inhibit activation of the cyclin B/Cdc2 kinase, which promotes mitotic entry, and abrogate G.sub.2 arrest induced by DNA damage inflicted by either anticancer agents or endogenous DNA damage, as well as result in preferential killing of the resulting checkpoint defective cells. See, e.g., Peng et al., Science, 277, 1501-1505 (1997); Sanchez et al., Science, 277, 1497-1501 (1997), Nurse, Cell, 91, 865-867 (1997); Weinert, Science, 277, 1450-1451 (1997); Walworth et al., Nature, 363, 368-371 (1993); and Al-Khodairy et al., Molec. Biol. Cell., 5, 147-160 (1994).

Selective manipulation of checkpoint control in cancer cells could afford broad utilization in cancer chemotherapeutic and radiotherapy regimens and may, in addition, offer a common hallmark of human cancer “genomic instability” to be exploited as the selective basis for the destruction of cancer cells. A number of factors place CHK1 as a pivotal target in DNA-damage checkpoint control. The elucidation of inhibitors of this and functionally related kinases such as CDS1/CHK2, a kinase recently discovered to cooperate with CHK1 in regulating S phase progression (see Zeng et al., Nature, 395, 507-510 (1998); Matsuoka, Science, 282, 1893-1897 (1998)), could provide valuable new therapeutic entities for the treatment of cancer.

Another group of kinases are the tyrosine kinases. Tyrosine kinases can be of the receptor type (having extracellular, transmembrane and intracellular domains) or the non-receptor type (being wholly intracellular). Receptor-type tyrosine kinases are comprised of a large number of transmembrane receptors with diverse biological activity. In fact, about 20 different subfamilies of receptor-type tyrosine kinases have been identified. One tyrosine kinase subfamily, designated the HER subfamily, is comprised of EGFR (HER1), HER2, HER3 and HER4. Ligands of this subfamily of receptors identified so far include epithelial growth factor, TGF-alpha, amphiregulin, HB-EGF, betacellulin and heregulin. Another subfamily of these receptor-type tyrosine kinases is the insulin subfamily, which includes INS-R, IGF-IR, IR, and IR-R. The PDGF subfamily includes the PDGF-alpha and beta receptors, CSFIR, c-kit and FLK-II. The FLK family is comprised of the kinase insert domain receptor (KDR), fetal liver kinase-1(FLK-1), fetal liver kinase-4 (FLK-4) and the fms-like tyrosine kinase-1 (fit-1). For detailed discussion of the receptor-type tyrosine kinases, see Plowman et al., DN&P 7(6): 334-339, 1994.

At least one of the non-receptor protein tyrosine kinases, namely, LCK, is believed to mediate the transduction in T-cells of a signal from the interaction of a cell-surface protein (Cd4) with a cross-linked anti-Cd4 antibody. A more detailed discussion of non-receptor tyrosine kinases is provided in Bolen, Oncogene, 8, 2025-2031 (1993). The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen, Oncogene, 8:2025-2031 (1993).

In addition to its role in cell-cycle control, protein kinases also play a crucial role in angiogenesis, which is the mechanism by which new capillaries are formed from existing vessels. When required, the vascular system has the potential to generate new capillary networks in order to maintain the proper functioning of tissues and organs. In the adult, however, angiogenesis is fairly limited, occurring only in the process of wound healing and neovascularization of the endometrium during menstruation. On the other hand, unwanted angiogenesis is a hallmark of several diseases, such as retinopathies, psoriasis, rheumatoid arthritis, age-related macular degeneration, and cancer (solid tumors). Protein kinases which have been shown to be involved in the angiogenic process include three members of the growth factor receptor tyrosine kinase family; VEGF-R2 (vascular endothelial growth factor receptor 2, also known as KDR (kinase insert domain receptor) and as FLK 1); FGF-R (fibroblast growth factor receptor); and TEK (also known as Tie-2).

VEGF-R2, which is expressed only on endothelial cells, binds the potent angiogenic growth factor VEGF and mediates the subsequent signal transduction through activation of its intracellular kinase activity. Thus, it is expected that direct inhibition of the kinase activity of VEGF-R2 will result in the reduction of angiogenesis even in the presence of exogenous VEGF (see Strawn et al, Cancer Research, 56, 3540-3545 (1996)), as has been shown with mutants of VEGF-R2 which fail to mediate signal transduction. Millauer et al., Cancer Research, 56, 1615-1620 (1996). Furthermore, VEGF-R2 appears to have no function in the adult beyond that of mediating the angiogenic activity of VEGF. Therefore, a selective inhibitor of the kinase activity of VEGF-R2 would be expected to exhibit little toxicity.

Similarly, FGFR binds the angiogenic growth factors aFGF and bFGF and mediates subsequent intracellular signal transduction. Recently, it has been suggested that growth factors such as bFGF may play a critical role in inducing angiogenesis in solid tumors that have reached a certain size. Yoshiji et al., Cancer Research, 57, 3924-3928 (1997). Unlike VEGF-R2, however, FGF-R is expressed in a number of different cell types throughout the body and may or may not play important roles in other normal physiological processes in the adult. Nonetheless, systemic administration of a small molecule inhibitor of the kinase activity of FGF-R has been reported to block bFGF-induced angiogenesis in mice without apparent toxicity. Mohammad et al., EMBO Journal, 17, 5996-5904 (1998).

TEK (also known as Tie-2) is another receptor tyrosine kinase expressed only on endothelial cells which has been shown to play a role in angiogenesis. The binding of the factor angiopoietin-1 results in autophosphorylation of the kinase domain of TEK and results in a signal transduction process which appears to mediate the interaction of endothelial cells with peri-endothelial support cells, thereby facilitating the maturation of newly formed blood vessels. The factor angiopoietin-2, on the other hand, appears to antagonize the action of angiopoietin-1 on TEK and disrupts angiogenesis. Maisonpierre et al., Science, 277, 55-60 (1997).

The kinase, JNK, belongs to the mitogen-activated protein kinase (MAPK) superfamily. JNK plays a crucial role in inflammatory responses, stress responses, cell proliferation, apoptosis, and tumorigenesis. JNK kinase activity can be activated by various stimuli, including the proinflammatory cytokines (TNF-alpha and interleukin-1), lymphocyte costimulatory receptors (CD28 and CD40), DNA-damaging chemicals, radiation, and Fas signaling. Results from the JNK knockout mice indicate that JNK is involved in apoptosis induction and T helper cell differentiation. Beta-carboline compounds and analogues as MK2 inhibitors are disclosed in WO2005/009370A2,A3, filed Jul. 22, 2004.

Pim-1 is a small serine/threonine kinase. Elevated expression levels of Pim-1 have been detected in lymphoid and myeloid malignancies, and recently Pim-1 was identified as a prognostic marker in prostate cancer. K. Peltola, “Signaling in Cancer: Pim-1 Kinase and its Partners”, Annales Uriiversitatis Turkuensis, Sarja-Ser. D. Osa-Tom. 616, (Aug. 30, 2005), http://kirjasto.utu.fi/julkaisupalvelut/annaalit/2004/D616.html. Pim-1 acts as a cell survival factor and may prevent apoptosis in malignant cells. K. Petersen Shay et al., Molecular Cancer Research 3:170-181 (2005).

Additionally, commonly owned, WO07/064,732, WO08/082490, and WO07/084,455 disclose compounds used to treat inflammatory disorders and are incorporated herein by reference.

There is a need for effective inhibitors of protein kinases in order to treat or prevent disease states associated with abnormal cell proliferation. Moreover, it is desirable for kinase inhibitors to possess both high affinity for the target kinase as well as high selectivity versus other protein kinases. Small-molecule compounds that may be readily synthesized and are potent inhibitors of cell proliferation are those, for example, that are inhibitors of one or more protein kinases, such as MK2, MK1, CHK1, CHK2, VEGF (VEGF-R2), Pim-1, CDKs or CDK/cyclin complexes and both receptor and non-receptor tyrosine kinases.

### SUMMARY OF THE INVENTION

In its many embodiments, the present invention provides a novel class of imidazo[1,2-a]pyrazine compounds, methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with protein kinases using such compounds or pharmaceutical compositions.

In one aspect, the present invention provides compounds selected from the group consisting of:

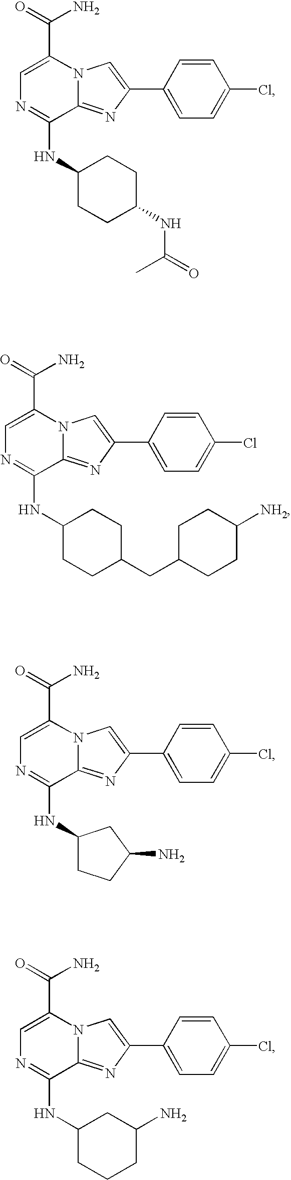

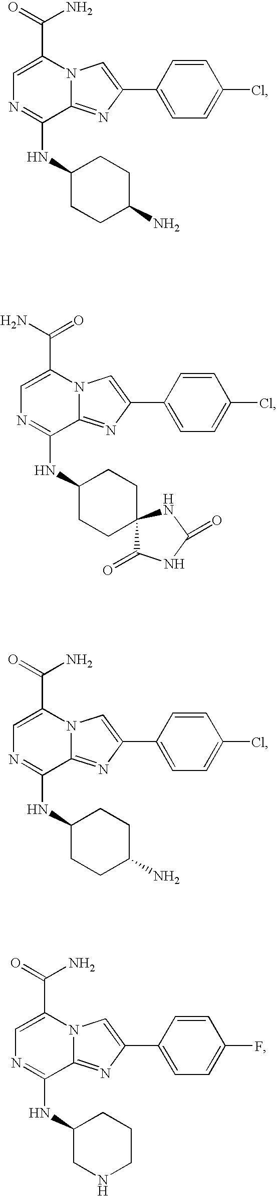

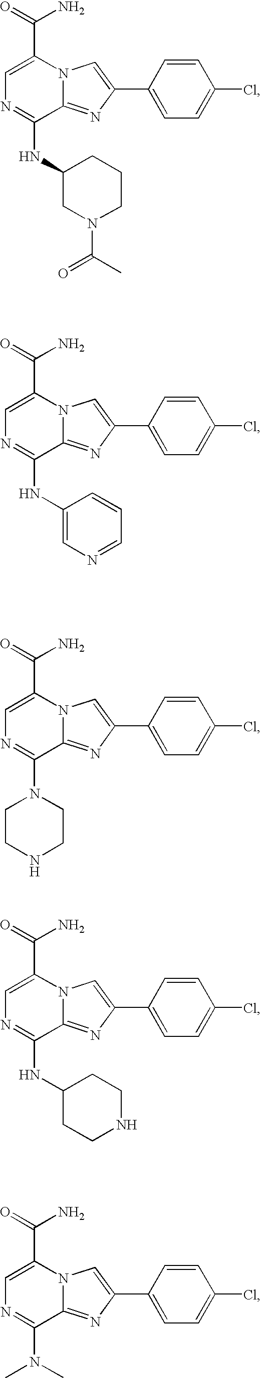

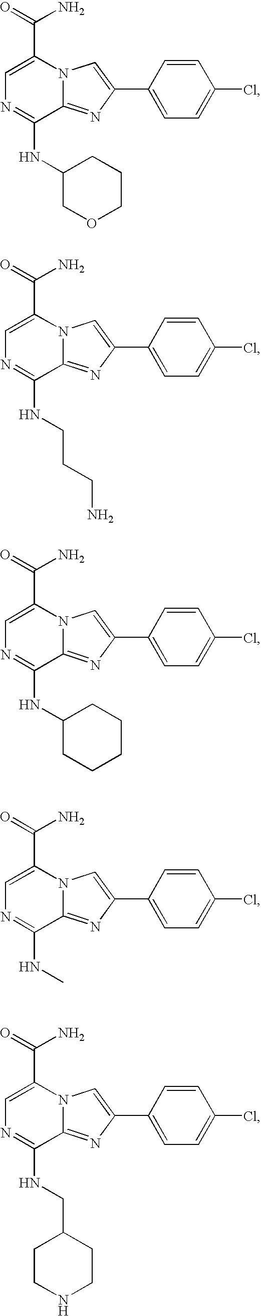

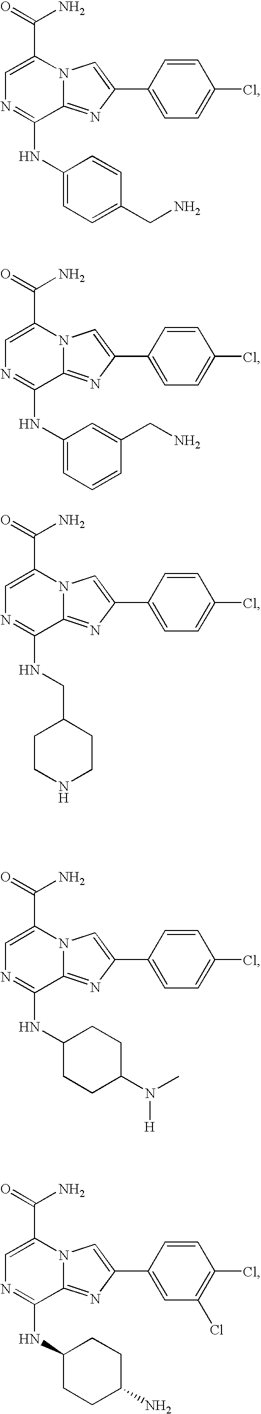

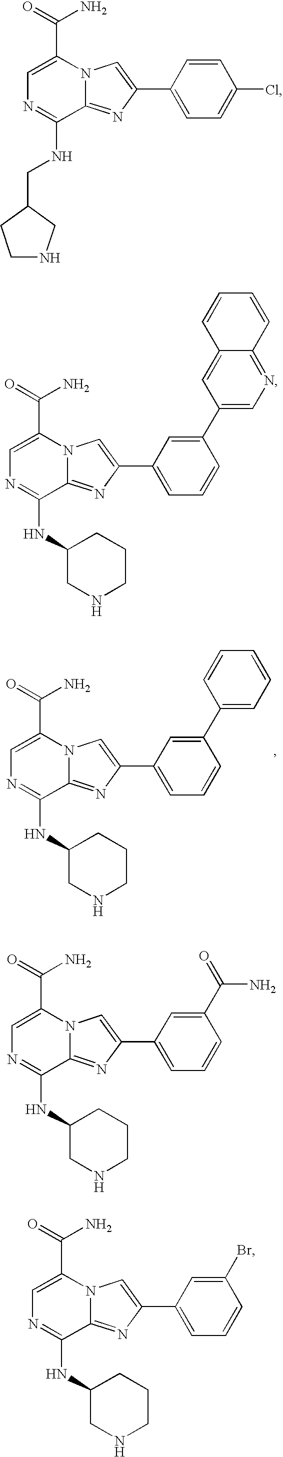

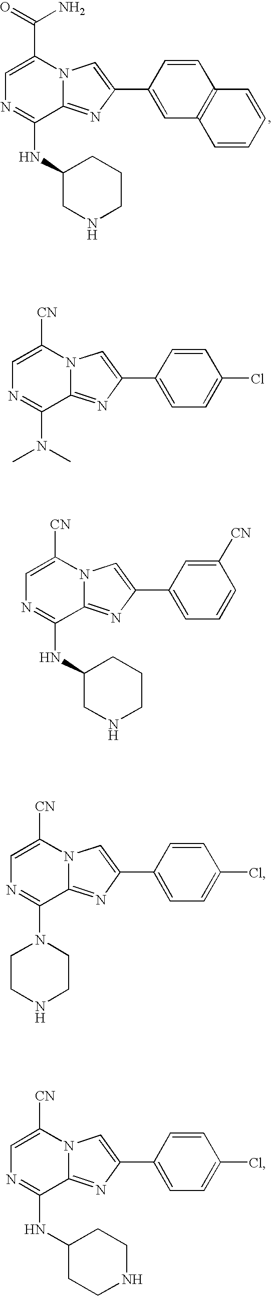

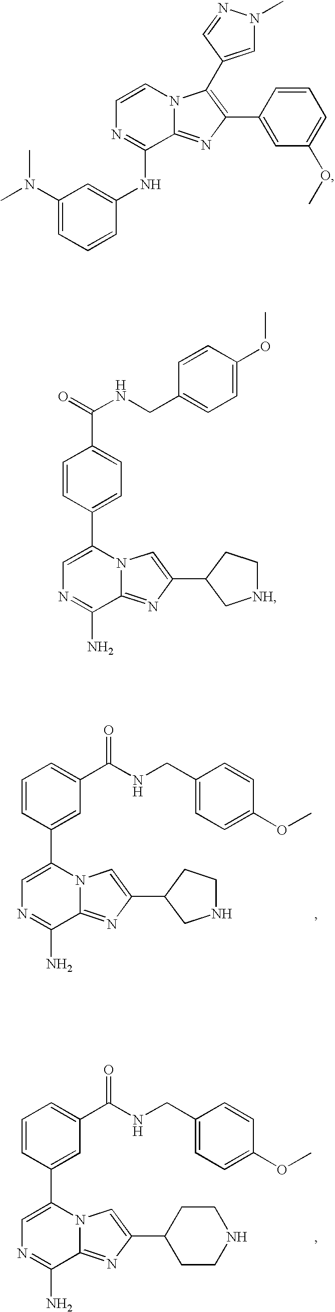

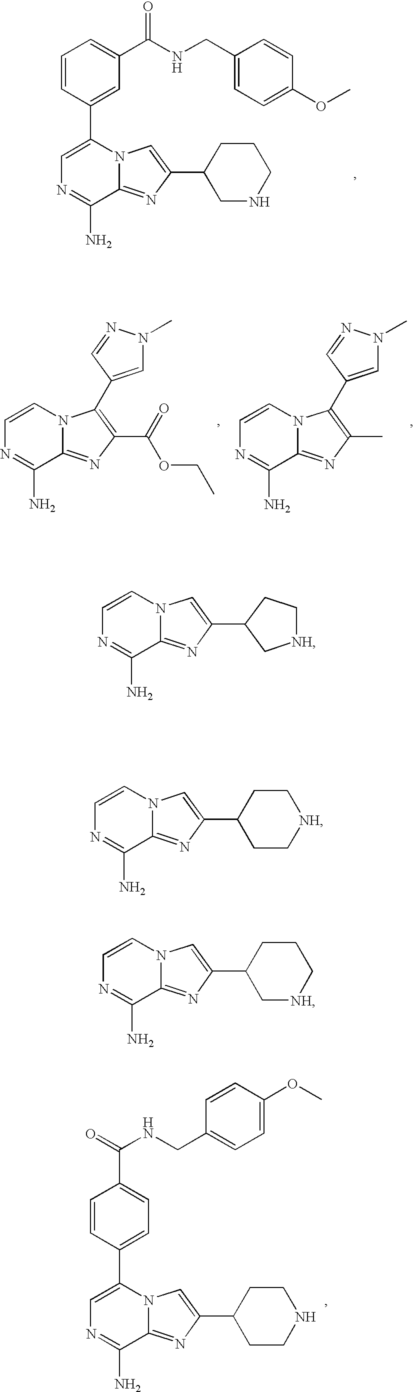

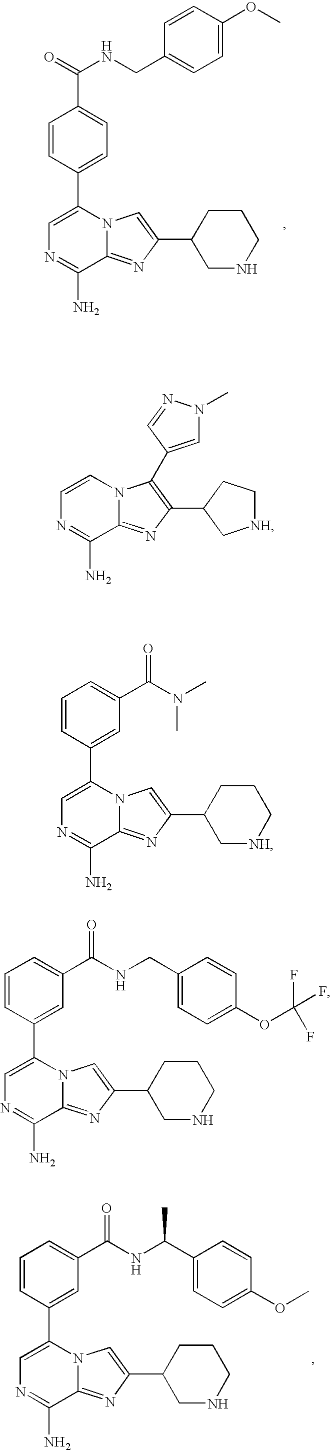

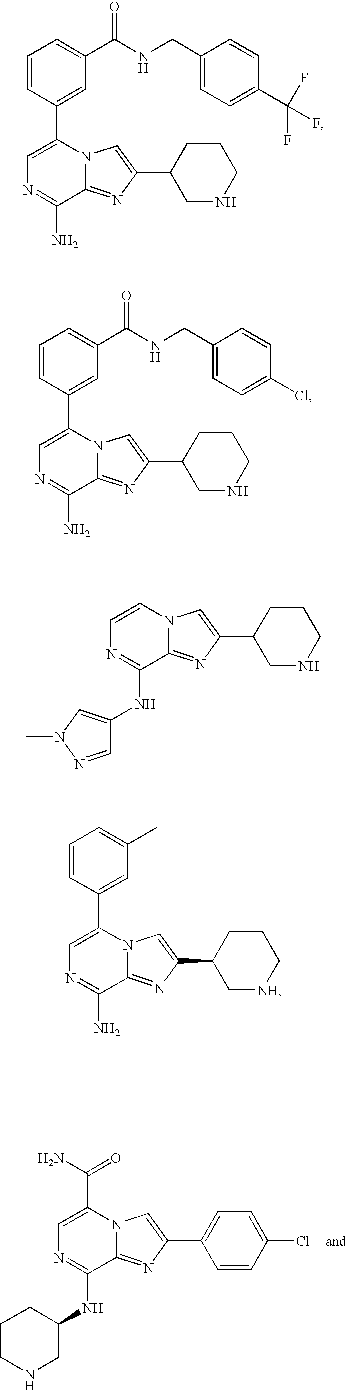

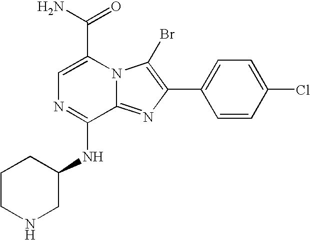

The compounds of the present invention can be useful as protein kinase inhibitors and can be useful in the treatment and prevention of proliferative diseases, for example, cancer, inflammation and arthritis, neurodegenerative diseases such Alzheimer's disease, cardiovascular diseases, viral diseases and fungal diseases.

### DETAILED DESCRIPTION

In an embodiment, the present invention provides imidazopyrazine compounds, especially imidazo[1,2-a]pyrazine compounds which are selected from the group of compounds listed above, or pharmaceutically acceptable salts, solvates, esters or prodrug thereof.

In another embodiment, the present invention provides for the use of compounds of Formula I:

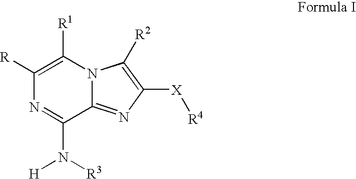

or a pharmaceutically acceptable salt, solvate, ester or prodrug thereof, wherein:
R is selected from the group consisting of H, halo, amino, alkyl, aryl, heteroaryl, heteroarylalkyl-, cycloalkyl, cycloalkylalkyl-, cycloalkenyl, cycloalkenylalkyl-, arylalkyl-, heterocyclyl, heterocyclylalkyl-, heterocyclenyl, heterocyclenylalkyl-, alkenyl-, alkynyl-, and —CN, wherein each of said alkyl, aryl, heteroaryl, heterocyclyl and cycloalkyl can be unsubstituted or optionally independently substituted with one or more moieties which can be the same or different each moiety being independently selected from halo, cyano, —N(R¹⁴R¹⁵), —C(O)—N(R¹⁴R¹⁵) and —N(R¹⁴)C(O)R¹⁵;
R¹ is selected from the group consisting of H, alkyl, aryl, arylalkyl-, heteroaryl, heteroarylalkyl-, cycloalkenyl, cycloalkenylalkyl, arylalkyl-, heterocyclyl, heterocyclylalkyl-, heterocyclenyl, heterocyclenylalkyl, and
   —C(O)—N(R¹⁴R¹⁵), wherein each of said alkyl, aryl, heteroaryl, heterocyclyl and cycloalkyl can be unsubstituted or optionally independently substituted with one or more moieties which can be the same or different each moiety being independently selected from halo, alkyl, aryl, cyano, —OR¹⁴, —SR¹⁴, —S(O)R¹⁴, —S(O₂)R¹⁴, —N(R¹⁴R¹⁵),
   —C(O)O-alkyl, —C(O)—N(R¹⁴R¹⁵), —N(R¹⁴)S(O₂)R¹⁵, and
   —N(R¹⁴)C(O)R¹⁵;
R² is selected from the group consisting of H, halo, amino, alkyl, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵) and —N(R¹⁴)C(O)R¹⁵;
R³ is selected from the group consisting of H, alkyl, amino, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, —C(O)N(R¹⁴R¹⁵) and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵) and —N(R¹⁴)C(O)R¹⁵;
X is selected from the group consisting of a covalent bond, —C(O)—NR¹⁴—S(O₂)N(R¹⁴)—, —N(R¹⁴)—C(O)—N(R¹⁵), —C(O)—O—, —O—C(O)—, —O—CO)—N(R¹⁴)—, —N(R¹⁴)—, —C(R¹⁴R¹⁵)—, —O—, —S—, —S(O)—, and —S(O₂)—;
   or the moiety

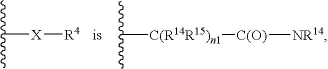

where n1 is 1-3;
R⁴ is selected from the group consisting of alkyl, —C(O)O-alkyl, heterocyclyl, aryl, and amino, wherein each of said alkyl, aryl and heterocyclyl can be unsubstituted or optionally independently substituted with one or more moieties which can be the same or different each moiety being independently selected from halo, alkyl, cyano, —N(R¹⁴R¹⁵),
   —C(O)—N(R¹⁴R¹⁵), —S(O₂)—N(R¹⁴R¹⁵), —S(O)—N(R¹⁴R¹⁵), —N(R¹⁴)C(O)R¹⁵, —C(O)O-alkyl, —O(CO)O-alkyl, —C(O)O-aryl, —C(O)O-heteroaryl, —OR¹⁴, —SR¹⁴, —O—CO—N(R¹⁴R¹⁵)— and —N(R¹⁴)—CO—N(R¹⁴R¹⁵);
R⁶ is H, alkyl, cycloalkyl or aryl, wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OCF₃, —CF₃, dialkylamino-, alkylthio-, alkylsulfonyl-, dialkylaminosulfonyl-, —NO₂, -Q(O)O-alkyl, —O(CO)O-alkyl, —C(O)O-aryl, and —C(O)O-heteroaryl;
R⁷ is H, alkyl, cycloalkyl or aryl wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OCF₃, —CF₃, dialkylamino-, alkylthio-, alkylsulfonyl-, dialkylaminosulfonyl-, —NO₂, —C(O)O-alkyl,
   —O(CO)O-alkyl, —C(O)O-aryl, and —C(O)O-heteroaryl; and
R¹⁴ and R¹⁵ can be the same or different, each being independently selected from the group consisting of H, alkyl, aryl, heteroaryl, arylalkyl, cycloalkyl, heterocyclyl, acyl, halo, hydroxyalkyl, trifluoromethyl, —OR⁶,
   —OCF₃, —N(R⁶R⁷), —SR⁶, —S(O₂)R⁶, —CN, —S(O₂)N(R⁶R⁷) and —NO₂₅ wherein each of said alkyl, aryl, heteroaryl, cycloalkyl and heterocyclyl can be unsubstituted or optionally independently substituted with one or more moieties which can be the same or different each moiety being independently selected from halo, alkyl, cyano, acyl, hydroxyalkyl, trifluoromethyl, —OR⁶, —OCF₃₁—CF₃, —N(R⁶R⁷), —SR⁶, —S(O₂)R⁶,
      —S(O₂)N(R⁶R⁷), —NO₂, —C(O)O-alkyl, —O(CO)O-alkyl, —C(O)O-aryl, and —C(O)O-heteroaryl;
         with the proviso that the invention excludes the following compounds:

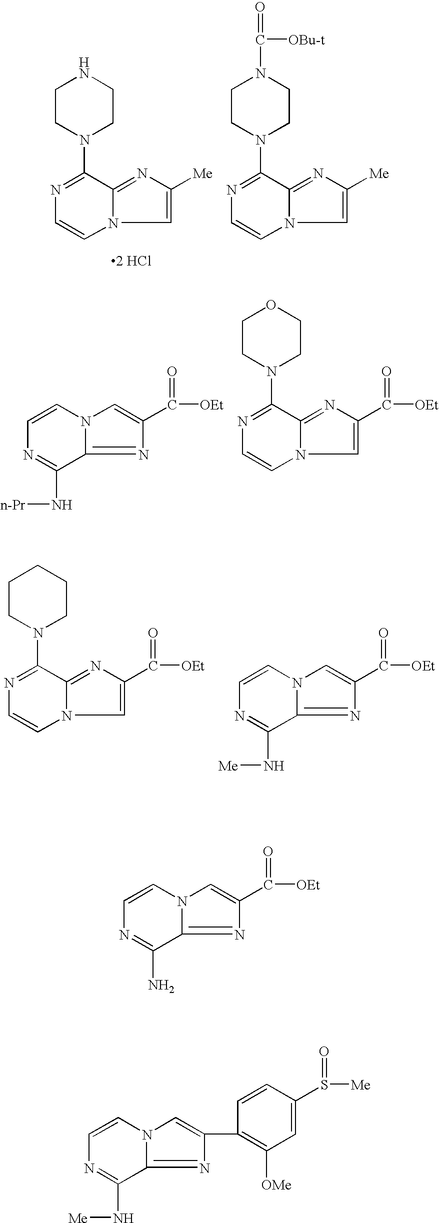

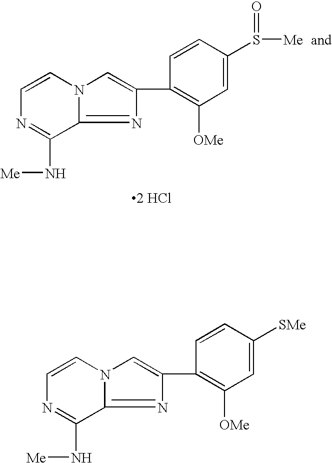

to inhibit one or more mitogen-activated protein kinase-activated protein kinase, or to treat one or more diseases associated with a mitogen-activated protein kinase-activated protein kinase.

In another embodiment, the present invention provides for the use of compounds of Formula II:

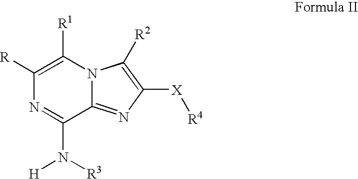

or a pharmaceutically acceptable salt, solvate, ester or prodrug thereof wherein:

R is H;

R¹ is CN or —C(O)—N(R¹⁴R¹⁵);

R² is H;

R³ is selected from the group consisting of alkyl, aryl, heteroaryl, cycloalkyl, cycloalkylalkyl-, heterocyclyl, and heterocyclylalkyl, wherein each of said alkyl, aryl, heteroaryl, cycloalkyl, cycloalkylalkyl-, heterocyclyl, and heterocyclylalkyl can be unsubstituted or optionally independently substituted with one or more moieties which can be the same or different each moiety being independently selected from, —N(R¹⁴R¹⁵), —C(O)—N(R¹⁴R¹⁵), —N(R¹⁴)C(O)R¹⁵ and spiroheterocyclyl group;

X is a covalent bond;

R⁴ is aryl, wherein said aryl can be unsubstituted or optionally independently substituted with one or more moieties which can be the same or different each moiety being independently selected from halo, cyano, —C(O)—N(R¹⁴R¹⁵), aryl and heteroaryl;

R⁵ is H or alkyl or R⁴ and R⁵ together with the N to which each is attached form a heterocyclyl,

R¹⁴ and R¹⁵ can be the same or different, each being H or alkyl to inhibit one or more mitogen-activated protein kinase-activated protein kinase, or to treat one or more diseases associated with a mitogen-activated protein kinase-activated protein kinase.

As used above, and throughout this disclosure, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. Lower alkyl refers to a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. Lower alkenyl refers to about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkenylene” means a difunctional group obtained by removal of a hydrogen from an alkenyl group that is defined above. Non-limiting examples of alkenylene include —CH═CH—, —C(CH₃)═CH—, and —CH═CHCH₂—.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. Lower alkynyl refers to about 2 to about 6 carbon atoms in the chain which may be straight or branched Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl dan be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

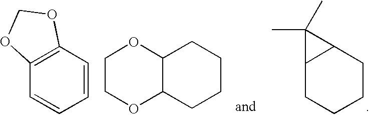

“Heteroarylalkyl” means a heteroaryl moiety as defined above linked via an, alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

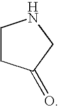

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

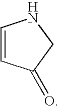

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

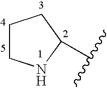

there is no —OH attached directly to carbons marked 2 and 5.

It should also be noted that tautomeric forms such as, for example, the moieties:

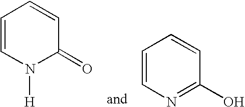

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O), alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkythio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group, Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of the present invention or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of the present invention or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of the present invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of the present invention incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N—(C₁-C₆)alkylaminoalkyl, —C(Y⁴)Y³ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate is a solvate wherein the solvent molecule is H₂O.

One or more compounds of the invention may optionally be converted to a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of the present invention can form salts which are also within the scope of this invention. Reference to a compound of the present invention herein is understood to include reference to salts thereof unless otherwise indicated. The term “salt(s)”, as employed herein, denotes acidic salts formed with inorganic and/or organic acids, as well as basic salts formed with inorganic and/or organic bases. In addition, when a compound of the present invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the present invention may be formed, for example, by reacting a compound of the present invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of the present invention, and salts, solvates, esters and prodrugs thereof may exist in their tautomeric form (for example, as an amide or imino ether). All such tautomeric forms are contemplated herein as part of the present invention.

The compounds of the present invention may contain asymmetric or chiral centers, and, therefore, exist in different stereoisomeric forms. It is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the present invention may be atropisomers (e.g., substituted biaryls) and are considered as part of this invention. Enantiomers can also be separated by use of chiral HPLC column.

It is also possible that the compounds of the present invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of the present invention incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of the present invention (e.g., those labeled with ³H and ¹⁴C) are useful in compound and/or substrate tissue distribution assays. Tritiated (i.e., 3H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the present invention can generally be prepared by following procedures analogous to those disclosed in the Schemes and/or in the Examples herein below, by substituting an appropriate isotopically labelled reagent for a non-isotopically labelled reagent.

Polymorphic forms of the compounds of the present invention, and of the salts, solvates, esters and prodrugs of the compounds of the present invention, are intended to be included in the present invention.

The compounds according to the invention have pharmacological properties; in particular, the compounds of the present invention can be inhibitors, regulators or modulators of protein kinases. Non-limiting examples of protein kinases that can be inhibited, regulated or modulated include mitogen activated protein kinase (MAPK/ERK) such as MK1, MK2, MAPKAP(k2,K3), cyclin-dependent kinases (CDKs), such as, CDK1, CDK2, CDK3, CDK4, CDK5, CDK6 and CDK7, CDK8, glycogen synthase kinase 3 (GSK3beta), Pim-1 kinases, Chk kinases, such as Chk1 and Chk2, tyrosine kinases, such as the HER subfamily (including, for example, EGFR (HER1), HER2, HER3 and HER4), the insulin subfamily (including, for example, INS-R, IGF-IR, IR, and IR-R), the PDGF subfamily (including, for example, PDGF-alpha and beta receptors, CSFIR, c-kit and FLK-II), the FLK family (including, for example, kinase insert domain receptor (KDR), fetal liver kinase-1 (FLK-1), fetal liver kinase-4 (FLK-4) and the fms-like tyrosine kinase-1 (fit-1)), non-receptor protein tyrosine kinases, for example LCK, Src, Frk, Btk, Csk, Abl, Zap70, Fes/Fps, Fak, Jak, Ack, and LIMK, growth factor receptor tyrosine kinases such as VEGF-R2, FGF-R, TEK, Akt kinases and the like.

The compounds of the present invention can be inhibitors of protein kinases such as, for example, the inhibitors of the mitogen-activated protein kinase-activated protein kinases (MAPKAPKs) such as MK1 and MK2 or the inhibitors of the checkpoint kinases such as Chk1, Chk2 and the like. Preferred compounds can exhibit IC₅₀ values of less than about 5 μm, preferably about 0.001 to about 1.0 μm, and more preferably about 0.001 to about 0.1 μm. The assay methods are described in the Examples set forth below.

The compounds of the present invention can be useful in the therapy of proliferative diseases such as cancer, autoimmune diseases, viral diseases, fungal diseases, neurological/neurodegenerative disorders, arthritis, inflammation, anti-proliferative (e.g., ocular retinopathy), neuronal, alopecia and cardiovascular disease. Many of these diseases and disorders are listed in U.S. Pat. No. 6,413,974 cited earlier, incorporated by reference herein.

More specifically, the compounds of the present invention can be useful in the treatment of a variety of cancers, including (but not limited to) the following: carcinoma, including that of the bladder, breast, colon, kidney, liver, lung, including small cell lung cancer, non-small cell lung cancer, head and neck, esophagus, gall bladder, ovary, pancreas, stomach, cervix, thyroid, prostate, and skin, including squamous cell carcinoma;

hematopoietic tumors of lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myetoma, and Burkett's lymphoma;

hematopoietic tumors of myeloid lineage, including acute and chronic myelogenous leukemias, myelodysplastic syndrome and promyelocytic leukemia;

tumors of mesenchymal origin, including fibrosarcoma and rhabdomyosarcoma;

tumors of the central and peripheral nervous system, including astrocytoma, neuroblastoma, glioma and schwannomas; and

other tumors, including melanoma, seminoma, teratocarcinoma, osteosarcoma, xenoderoma pigmentosum, keratoctanthoma, thyroid follicular cancer and Kaposi's sarcoma.

Due to the key role of CDKs in the regulation of cellular proliferation in general, inhibitors could act as reversible cytostatic agents which may be useful in the treatment of any disease process which features abnormal cellular proliferation, e.g., benign prostate hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.

Compounds of the present invention may also be useful in the treatment of Alzheimer's disease, as suggested by the recent finding that CDK5 is involved in the phosphorylation of tau protein (J. Biochem, (1995) 117, 741-749).

Compounds of the present invention may induce or inhibit apoptosis. The apoptotic response is aberrant in a variety of human diseases. Compounds of the present invention, as modulators of apoptosis, will be useful in the treatment of cancer (including but not limited to those types mentioned hereinabove), viral infections (including but not limited to herpevirus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis and arthritis) aspirin-sensitive rhinosinusitis, cystic fibrosis, multiple sclerosis, kidney diseases and cancer pain.

Compounds of the present invention, as inhibitors of the CDKs, can modulate the level of cellular RNA and DNA synthesis. These agents would therefore be useful in the treatment of viral infections (including but not limited to HIV, human papilloma virus, herpesvirus, poxvirus, Epstein-Barr virus, Sindbis virus and adenovirus).

Compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

Compounds of the present invention may also be useful in inhibiting tumor angiogenesis and metastasis.

Compounds of the present invention, as inhibitors of MKs, can decrease inflammation, these agents would therefore be useful in the treatment of various inflammatory diseases, including, but not limited to rheumatoid arthritis, osteoarthritis, periodontitis, gingivitis, corneal ulceration, solid tumor growth and tumor invasion by secondary metastases, neovascular glaucoma, inflammatory bowel disease, multiple sclerosis and psoriasis.

Compounds of the present invention may be useful as novel inhibitors of protein kinases selected from the group consisting of MAPK(1,2), MAPKAP(k2,k3), CHK (1 and 2), CDK (1, 2, 3, 4, 5, 6, 7), HIPK(1,2,3) JNK (1,2,3), Lck, MEK (1), ERK (1, 2), c-Met, c-Kit, Aurora (A, B), ARK5, IKK(α,β), MSK1, NEK(2,3,6,7,11), Pim(1,2,3), Plk3, PAK family (Pak, 1,2,3,4,5,6), Rock I, II, Ron, Ros, SGK (1, 2, 3), IKK-beta, m-TOR, IRAK(1,4), COT kinase, CLK3, BRaf, INSR, AKT (1,2,3), TAK1, mTOR, AMPK(1,2), PAR1B-a, WNK(2,3), ZAP 70, and the receptor.

The non-receptor type of tyrosine kinases is also comprised of numerous subfamilies, including Src, Frk, Btk, Csk, Abl, Zap70, Fes/Fps, Fak, Jak, Ack, and LIMK, SRC subfamily (Src, Yes, Fyn, Lyn, Lck, Blk, Hck, Fgr, and Yrk), ALK4, AXL, BMX, Brk, EphB4, Fer, Fgr, JAK (1, 2,3), PDK (1), PKD 2, Rsk(1,2,3,4) Ret, TrkA, VEGFR, EGFR(HER1,2,3,4), FGFR(1,2,3,4), IGFR, PDGFR(α,β), GSK3(α,β), FLK(1,4), KDR, Flt(1,2,3,4), SRC, JAK and TEK families (Tie2), which are useful for the treatment of proliferative diseases, such as cancer.

In particular these inhibitors are useful to inhibit, MAPKAP-2 (MK2) and its isoforms and CHK kinases.

VEGFR, EGFR, HER2, SRC, CHK, JAK and TEK families are useful for the treatment of proliferative diseases such as cancer, while MAPKAP-2, IKK and IRAK kinases inhibitors are useful in treating inflammatory diseases such as Rhumetoid/osteo arthritis.

Compounds of the present invention may also act as inhibitors of other protein kinases, e.g., protein kinase C, her2, raf 1, MEK1, MAP kinase, EGF receptor, PDGF receptor, IGF receptor, PI3 kinase, wee1 kinase, Src, Abl and thus be effective in the treatment of diseases associated with other protein kinases.

Another aspect of this invention is a method of treating a mammal (e.g., human) having a disease or condition associated with the MKs or CHKs by administering a therapeutically effective amount of at least one compound of the present invention, or a pharmaceutically acceptable salt, solvate, ester or prodrug of said compound to the mammal.

A preferred dosage is about 0.001 to 500 mg/kg of body weight/day of the compound of the present invention. An especially preferred dosage is about 0.01 to 25 mg/kg of body weight/day of a compound of the present invention, or a pharmaceutically acceptable salt, solvate, ester or prodrug of said compound.

The compounds of this invention may also be useful in combination (administered together, concurrently, as fixed dose, or sequentially) with one or more of anti-cancer treatments such as radiation therapy, and/or one or more anti-cancer agents different from the compounds of the present invention. The compounds of the present invention can be present in the same dosage unit as the anti-cancer agent or in separate dosage units.

Another aspect of the present invention is a method of treating one or more diseases associated with mitogen-activated protein kinase-activated protein kinase, comprising administering to a mammal in need of such treatment an amount of a first compound, which is a compound of the present invention, or a pharmaceutically acceptable salt, solvate, ester or prodrug thereof; and an amount of at least one second compound, the second compound being an anti-cancer agent different from the compound of the present invention, wherein the amounts of the first compound and the second compound result in a therapeutic effect.

Non-limiting examples of suitable anti-cancer agents include cytostatic agents, cytotoxic agents (such as for example, but not limited to, DNA interactive agents (such as cisplatin or doxorubicin)); taxanes (e.g. taxotere, taxol); topoisomerase II inhibitors (such as etoposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methoxtrexate); alkylating agents (such as temozolomide (TEMODAR™ from Schering-Plough Corporation, Kenilworth, N.J.), cyclophosphamide); Farnesyl protein transferase inhibitors (such as, SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-1-piperidinecarboxamide, or SCH 66336 from Schering-Plough Corporation, Kenilworth, N.J.), tipifarnib (Zarnestra® or R115777 from Janssen Pharmaceuticals), L778123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.); signal transduction inhibitors (such as, Iressa® (or gefitinib from Astra Zeneca Pharmaceuticals, England), Tarceva® (erlotinib hydrochloride) (EGFR kinase inhibitors), antibodies to EGFR (e.g., C225), GLEEVEC® (imatinib, a C-abl kinase inhibitor from Novartis Pharmaceuticals, East Hanover, N.J.); interferons such as, for example, intron (from Schering-Plough Corporation), Peg-Intron (from Schering-Plough Corporation); hormonal therapy combinations; aromatase combinations; ara-C, adriamycin, cytoxan, and gemcitabine.

Other anti-cancer (also known as anti-neoplastic) agents include but are not limited to Uracil mustard, Chlormethine, Ifosfamide, Melphalan, Chlorambucil, Pipobroman, Triethylenemelamine, Busulfan, Carmustine, Lomustine, triethylenethiophosphoramine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaceuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux® (cetuximab from Bristol-Myers Squibb), Liposomal, Thiotepa, Altretamine, Melphalan, Trastuzumab, Lerozole, Fulvestrant, Exemestane, Ifosfomide, Rituximab, C225 (from Merck KGaA, Darmstadt, Germany), Doxil (from Johnson & Johnson, New Brunswick, N.J.), Ontak (from Seragen), Depocyt (from SkyePharma), Mylotarg (from Wyeth Pharmaceuticals), Campath (from Genzyme/Ilex Technology), Celebrex (from Pfizer, New York, N.Y.), Sutent (from Pfizer), Aranesp (from Amgen), Neupogen (from Amgen), Neulasta (from Amgen), Kepivance (from Amgen), SU11248 and PTK787.

Other anti-inflammatory agents include but are not limited to disease modifying anti-rheumatic drugs (DMARDS), non-steroidal anti-inflammatory drugs (NSAIDs), cycloxygenase-2 selective (COX-2) inhibitors, COX-1 inhibitors, immunosuppressives, biological response modifiers (BRMs), anti-inflammatory agents and H₁ antagonists.

If formulated as a fixed dose, such combination products employ the compounds of this invention within the dosage range described herein and the other pharmaceutically active agent or treatment within its dosage range. For example, the CDC2 inhibitor olomucine has been found to act synergistically with known cytotoxic agents in inducing apoptosis (J. Cell Sci., (1995) 108, 2897. Compounds of the present invention may also be administered sequentially with known anticancer or cytotoxic agents when a combination formulation is inappropriate. The invention is not limited in the sequence of administration; compounds of the present invention may be administered either prior to or after administration of the known anticancer or cytotoxic agent. For example, the cytotoxic activity of the cyclin-dependent kinase inhibitor flavopiridol is affected by the sequence of administration with anticancer agents. Cancer Research, (1997) 57, 3375. Such techniques are within the skills of persons skilled in the art as well as attending physicians.

Accordingly, in an aspect, this invention includes combinations comprising an amount of at least one compound of the present invention, or a pharmaceutically acceptable salt, solvate, ester or prodrug thereof, and an amount of one or more anti-cancer treatments and anti-cancer agents listed above wherein the amounts of the compounds/treatments result in desired therapeutic effect.

Another aspect of this invention includes combinations comprising an amount of at least one compound of the present invention, or a pharmaceutically acceptable salt, solvate, ester or prodrug thereof, and an amount of one or more anti-inflammatory treatments and anti-inflammatory agents listed above wherein the amounts of the compounds/treatments result in desired therapeutic effect.

Another aspect of this invention is a method of inhibiting one or more mitogen-activated protein kinase-activated protein kinase, or treating one or more diseases associated with a mitogen-activated protein kinase-activated protein kinase, comprising administering a therapeutically effective amount of at least one compound of the present invention or a pharmaceutically acceptable salt, solvate, ester or prodrug thereof to a patient in need of such inhibition.

Still another aspect of this invention is a method of treating one or more diseases associated with mitogen-activated protein kinase-activated protein kinase, comprising administering to a mammal in need of such treatment

an amount of a first compound, which is a compound of the present invention, or a pharmaceutically acceptable salt, solvate, ester or prodrug thereof and

an amount of at least one second compound, the second compound-being an anti-cancer agent different from the compounds of the present invention;

wherein the amounts of the first compound and the second compound result in a therapeutic effect.

Even further still another aspect of the invention is a method of treating one or more diseases associated with mitogen-activated protein kinase-activated protein kinase, comprising administering to a mammal in need of such treatment

an amount of a first compound, which is a compound of the present invention, or a pharmaceutically acceptable salt, solvate, ester or prodrug thereof and

an amount of at least one second compound, the second compound being an anti-inflammatory agent different from the present invention;

wherein the amounts of the first compound and the second compound result in a therapeutic effect.

In the above methods, the mitogen-activated protein kinase-activated protein kinase to be inhibited can be MK1 and/or MK2.

Another aspect of this invention is a method of inhibiting one or more Checkpoint kinases in a patient in need thereof, comprising administering to the patient a therapeutically effective amount of at least one of the present invention or a pharmaceutically acceptable salt, solvate, ester or prodrug thereof.

Another aspect of the present invention is a method of treating, or slowing the progression of, a disease associated with one or more Checkpoint kinases in a patient in need thereof, comprising administering a therapeutically effective amount of at least one compound of the present invention or a pharmaceutically acceptable salt, solvate, ester or prodrug thereof.

Yet another aspect of the present invention is a method of treating one or more diseases associated with Checkpoint kinase, comprising administering to a mammal in need of such treatment an amount of a first compound, which is a compound of the present invention, or a pharmaceutically acceptable salt, solvate, ester or prodrug thereof; and an amount of at least one second compound, the second compound being an anti-cancer agent, wherein the amounts of the first compound and the second compound result in a therapeutic effect.

Another aspect of the present invention is a method of treating, or slowing the progression of, a disease associated with one or more Checkpoint kinases in a patient in need thereof, comprising administering a therapeutically effective amount of a pharmaceutical composition comprising in combination at least one pharmaceutically acceptable carrier and at least one compound of the present invention, or a pharmaceutically acceptable salt, solvate, ester or prodrug thereof.

In the above methods, the checkpoint kinase to be inhibited can be Chk1 and/or Chk2.

Another aspect of this invention is a method of treating a mammal (e.g., human) having a disease or condition associated with the CDKs by administering a therapeutically effective amount of at least one compound of the present invention, or a pharmaceutically acceptable salt, solvate, ester or prodrug of said compound to the mammal.

In the above methods, the cyclin dependent kinase to be inhibited can be CDK1 and/or CDK2.

Another aspect of the present invention is a method of inhibiting one or more tyrosine kinases in a patient in need thereof, comprising administering to the patient a therapeutically effective amount of at least one compound of the present invention or a pharmaceutically acceptable salt, solvate, ester or prod rug thereof.

Yet another aspect of the present invention is a method of treating, or slowing the progression of, a disease associated with one or more tyrosine kinases in a patient in need thereof, comprising administering a therapeutically effective amount of at least one compound of the present invention or a pharmaceutically acceptable salt, solvate, ester or prod rug thereof.

Another aspect of the present invention is a method of treating one or more diseases associated with tyrosine kinase, comprising administering to a mammal in need of such treatment an amount of a first compound, which is a compound of the present invention, or a pharmaceutically acceptable salt, solvate, ester or prodrug thereof; and an amount of at least one second compound, the second compound being an anti-cancer agent, wherein the amounts of the first compound and the second compound result in a therapeutic effect.

Another aspect of the present invention is a method of treating, or slowing the progression of, a disease associated with one or more tyrosine kinases in a patient in need thereof, comprising administering a therapeutically effective amount of a pharmaceutical composition comprising in combination at least one pharmaceutically acceptable carrier and at least one compound of the present invention or a pharmaceutically acceptable salt, solvate, ester or prodrug thereof.

In the above methods, the tyrosine kinase can be VEGFR (VEGF-R2), EGFR, HER2, SRC, JAK and/or TEK.

Another aspect of the present invention is a method of inhibiting one or more Pim-1 kinases in a patient in need thereof, comprising administering to the patient a therapeutically effective amount of at least one compound of the present invention or a pharmaceutically acceptable salt, solvate, ester or prodrug thereof.

Yet another aspect of the present invention is a method of treating, or slowing the progression of, a disease associated with one or more Pim-1 kinases in a patient in need thereof, comprising administering a therapeutically effective amount of at least one compound of the present invention or a pharmaceutically acceptable salt, solvate, ester or prodrug thereof.

Another aspect of the present invention is a method of treating one or more diseases associated with Pim-1 kinase, comprising administering to a mammal in need of such treatment an amount of a first compound, which is a compound of the present invention or a pharmaceutically acceptable salt, solvate, ester or prodrug thereof; and an amount of at least one second compound, the second compound being an anti-cancer agent, wherein the amounts of the first compound and the second compound result in a therapeutic effect.

Another aspect of the present invention is a method of treating, or slowing the progression of, a disease associated with one or more Pim-1 kinases in a patient in need thereof, comprising administering a therapeutically effective amount of a pharmaceutical composition comprising in combination at least one pharmaceutically acceptable carrier and at least one compound of the present invention or a pharmaceutically acceptable salt, solvate, ester or prodrug thereof.

The pharmacological properties of the compounds of this invention may be confirmed by a number of pharmacological assays. The exemplified pharmacological assays which are described herein below have been carried out with compounds according to the invention and their salts, solvates, esters or prodrugs.

This invention is also directed to pharmaceutical compositions which comprise at least one compound of the present invention, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally or intravenously or intrathecally or some suitable combination(s) thereof.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of the present invention, or a pharmaceutically acceptable salt, solvate, ester or prodrug of said compound and a pharmaceutically acceptable carrier, vehicle or diluent.

Yet another aspect of this invention is a kit comprising an amount of at least one compound of the present invention, or a pharmaceutically acceptable salt, solvate, ester or prodrug of said compound and an amount of at least one anticancer therapy and/or anti-cancer agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art.

Where NMR data are presented, ¹H spectra were obtained on a Varian AS-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The retention time and observed parent ion are given.

The following solvents and reagents may be referred to by their abbreviations in parenthesis:

Thin layer chromatography: TLC
dichloromethane: CH₂Cl₂
ethyl acetates: AcOEt or EtOAc
methanol: MeOH
trifluoroacetate: TFA
triethylamine: Et₃N or TEA
butoxycarbonyl: n-Boc or Boc
nuclear magnetic resonance spectroscopy: NMR
liquid chromatography mass spectrometry: LCMS
high resolution mass spectrometry: HRMS
milliliters: mL
millimoles: mmol
microliters: μl
grams: g
milligrams: mg
room temperature or rt (ambient): about 25° C.
dimethoxyethane: DME

The synthesis of the inventive compounds is illustrated below. Also, it should be noted that the disclosure of commonly-owned U.S. Pat. No. 6,919,341 is incorporated herein by reference.

### Synthesis

### Example 1

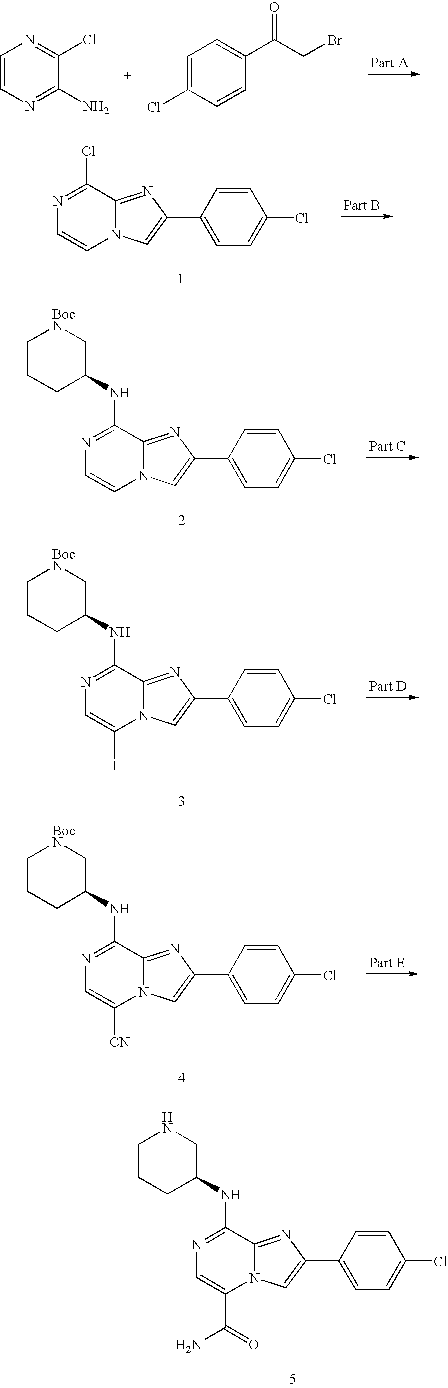

### Part A:

To a suspension of 2-bromo-1-(4-chlorophenyl)ethanone (7.21 g, 30.9 mmol) in DME (60 mL) was added 2-aminopyrazine (2.00 g, 15.4 mmol). The mixture was heated up to 90° C. and stirred for 1 day. After cooling to room temperature, the reaction mixture was taken with EtOAc and washed with NaHCO₃ (sat.aq.). After drying over Na₂SO₄ and concentration, EtOAc was added to the residue. The suspension was filtered and gave the product 1 (1.43 g) as solid. HPLC-MS t_{R}=2.00 min (UV_{254 nm}); mass calculated for formula C₁₂H₇Cl₂N₃ 263.0, observed LCMS m/z 264.0 (M+H).

### Part B:

The compound 1 (364 mg, 1.38 mmol) from part A was dissolved in NMP (3 ml) and amine (390 mg, 1.95 mmol), K₂CO₃ (230 mg, 1.67 mmol) were added. The mixture was heated up to 100° C. and stirred for 1 day. After cooling to room temperature, the mixture was diluted with EtOAc and washed with water and brine. After concentration, the residue was purified with column (silica get, EtOAc/Hexane=50/50) gave the product 2 (225 mg) as solid. HPLC-MS t_{R}=2.14 min (UV_{254 nm}); mass calculated for formula C22H₂₆ClN₅O₂ 427.2, observed LCMS m/z 428.2 (M+H).

### Part C:

To a solution of compound 2 (225 mg, 0.524 mmol) in CHCl₃ (3 mL), NIS (200 mg, 0.890 mmol) was added at room temperature. The mixture was stirred for 1.5 hr and then diluted with EtOAc and washed with Na₂S₂O₃ (sat. aq. 5 mL×2) and dried over Na₂SO₄. After concentration, the residue was purified with column (silica gel, EtOAc/Hexane=25/75) and gave the product 3 (180 mg) as an oil. HPLC-MS t_{R}=2.78 min (UV_{254 nm}); mass calculated for formula C22H₂₅IClN₅O₂ 553.1, observed LCMS m/z 554.1 (M+H).

### Part D:

A sealed tube was charged with compound 3 (180 mg, 0.32 mmol), Zn(CN)₂ (76 mg, 0.65 mmol) and Pd(PPh₃)₄ (55 mg, 0.05 mmol). DMF (2 mL) was added and the mixture was thoroughly degassed by bubbling Argon into the reaction mixture. This reaction was then heated at 80° C. overnight. After cooling to room temperature, it was concentrated to ⅔ its volume and purified with column (silica gel, EtOAc/hexane=25/75) to give the product 4 (118 mg) as oil. HPLC-MS t_{R}=2.58 min (UV_{254 nm}); mass calculated for formula C23H₂₅ClN₆O₂ 452.2, observed LCMS m/z 453.2 (M+H).

### Part E:

To the compound 4 (20 mg) in a 20 mL vial, H₂SO₄ (0.25 mL) was added and the resulting mixture was stirred for 1 hour at room temperature. Then, ice (2 g) was added and the pH was adjusted to 9 with 6N NaOH. The pH was then adjusted to 1 with 1 N HCl and then treated with NaHCO₃ (sat. aq.) to a pH of 8. The aqueous mixture was extracted with 20% iPrOH/CH₂Cl₂ (10 mL×3) and dried over Na₂SO₄. After concentration, the solid was purified with Prep-LC and gave the product 5. HPLC-MS t_{R}=2.70 min (UV_{254 nm}); mass calculated for formula C18H₁₉ClN₆O 370. 1, observed LCMS m/z 371.1 (M+H).

By essentially the same procedure given in Preparative Example 1, compounds 6-29 given in Column 2 of Table 1 can be prepared.

TABLE 1

| | | | MS | |
|---|---|---|---|---|
| | | Exact | m/z | HPLC |
| Example | Structure | mass | (M + H) | MS t_{R} |
| 6 | 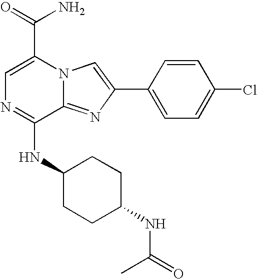 | 426.2 | 427.2 | 3.46 |
| 7 | 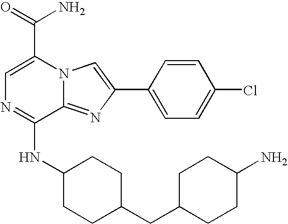 | 480.2 | 481.2 | 3.68 |
| 8 | 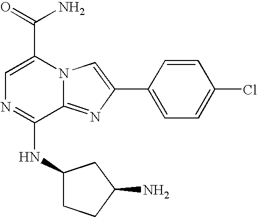 | 370.1 | 371.1 | 2.90 |
| 9 | 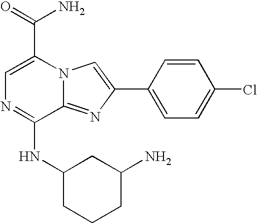 | 384.1 | 385.1 | 2.50 |
| 10 | 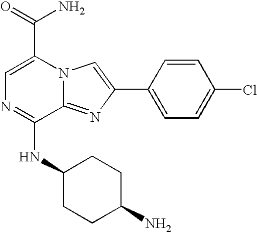 | 384.1 | 385.1 | 2.31 |
| 11 | 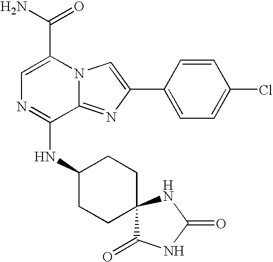 | 453.1 | 454.1 | 2.65 |
| 12 | 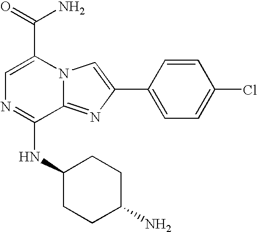 | 384.1 | 385.1 | 2.39 |
| 13 | 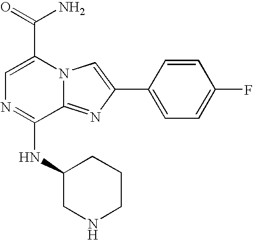 | 354.2 | 355.2 | 2.35 |
| 14 | 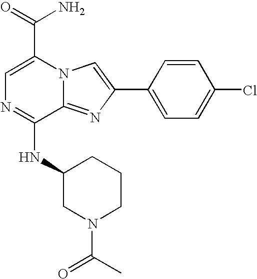 | 412.1 | 413.1 | 3.37 |
| 15 | 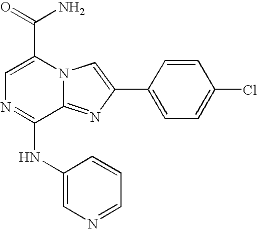 | 364.1 | .65.1 | 2.88 |
| 16 | 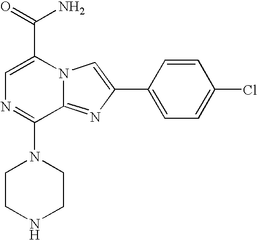 | 356.1 | 357.1 | 2.88 |
| 17 | 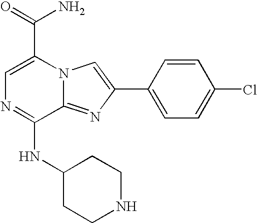 | 370.1 | 371.1 | 2.65 |
| 18 | 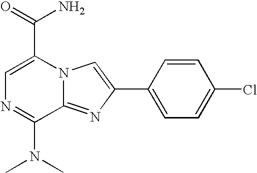 | 315.1 | 316.1 | 4.59 |
| 19 | 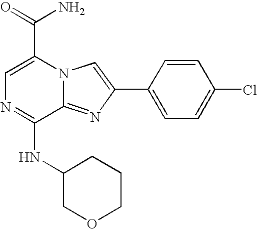 | 371.1 | 372.1 | 3.51 |
| 20 | 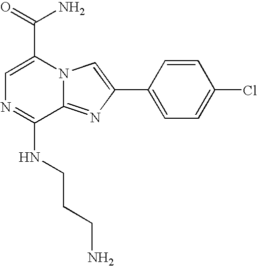 | 344.1 | 345.1 | 2.41 |
| 21 | 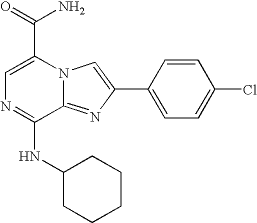 | 369.1 | 370.1 | 3.79 |
| 22 | 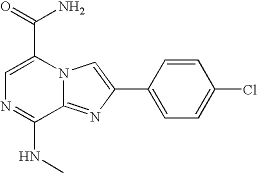 | 301.1 | 302.1 | 2.65 |
| 23 | 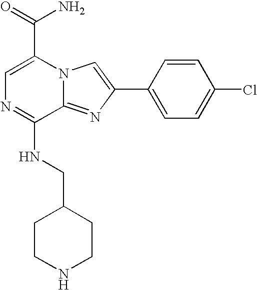 | 384.1 | 385.1 | 2.89 |
| 24 | 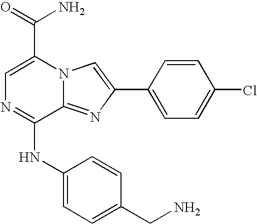 | 392.1 | 393.1 | 3.38 |
| 25 | 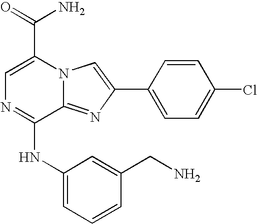 | 392.1 | 393.1 | 3.42 |
| 26 | 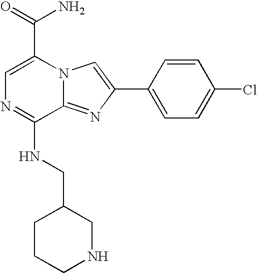 | 384.1 | 385.1 | 3.04 |
| 27 | 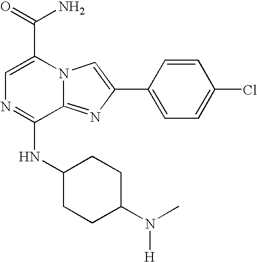 | 398.2 | 399.2 | 2.94 |
| 28 | 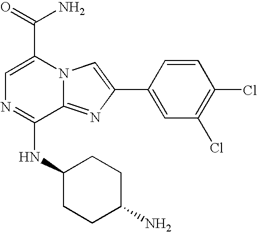 | 418.1 | 419.1 | 3.40 |
| 29 | 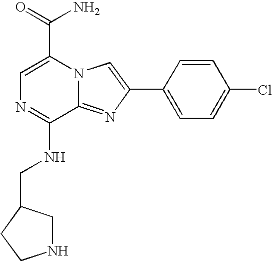 | 370.1 | 371.1 | |

### Example 2

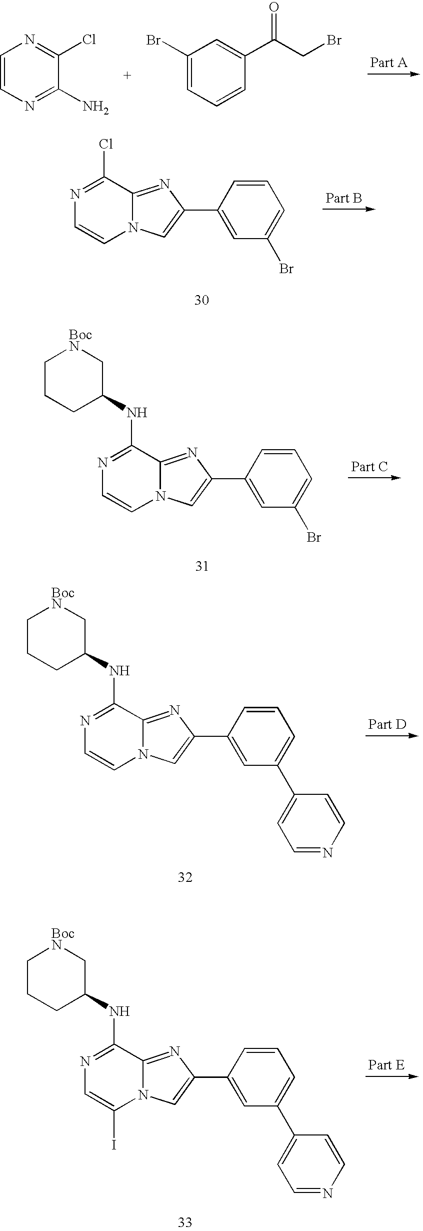

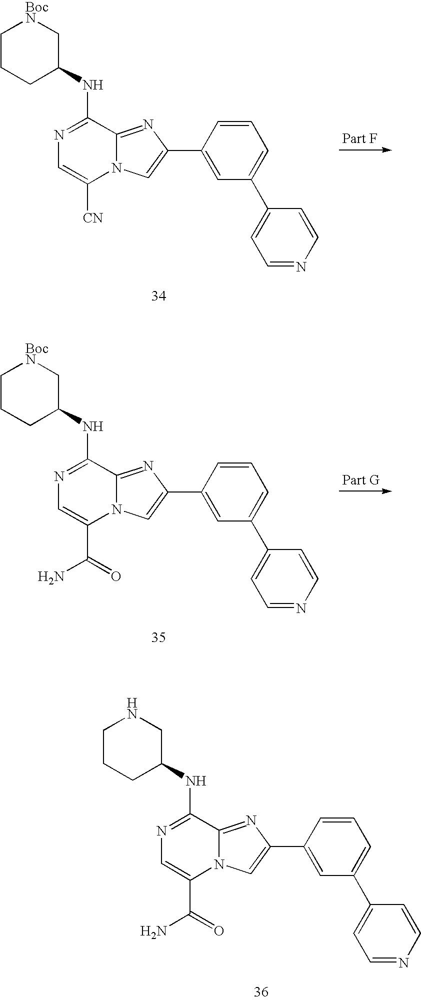

### Part A:

By essentially the same procedure given in Preparative Example 1, Part A, compound 30 can be prepared from 2-aminopyrazine and 2-bromo-1-(3-bromophenyl)ethanone. HPLC-MS t_{R}=2.06 min (UV_{254 nm}); mass calculated for formula C₁₂H₇BrClN₃ 307.0, observed LCMS m/z 308.0 (M+H).

### Part B:

By essentially the same procedure given in Preparative Example 1, Part B, compound 31 can be prepared. HPLC-MS t_{R}=2.02 min (UV_{254 nm}); mass calculated for formula C22H₂₆BrN₅O₂ 471.1, observed LCMS m/z 472.1 (M+H).

### Part C:

A sealed tube was charged with compound 31 (138 mg, 0.29 mmol), pyridin-4-ylboronic acid (72 mg, 0.58 mmol), K₃PO₄.H₂O (200 mg, 0.87 mmol), and PdCl₂(dppf) (35 mg, 0.044 mmol). Dioxane (3 mL) and H₂O (0.3 mL) % were added and the mixture was thoroughly degassed by bubbling Argon into the reaction mixture. This reaction was then heated at 85° C. overnight. After cooling to room temperature, it was diluted with EtOAc and washed with NaHCO₃ (sat. aq.). After concentration the residue was purified with column (silica gel, EtOAc/hexane=50150) to give the product 32 (75 mg) as oil. HPLC-MS t_{R}=1.43 min (UV_{254 nm}); mass calculated for formula C27H₃₀N₆O₂ 470.2, observed LCMS m/z 471.2 (M+H).

### Part D:

By essentially the same procedure given in Preparative Example 1, Part C, compound 33 can be prepared. HPLC-MS t_{R}=1.86 min (UV_{254 nm}); mass calculated for formula C₂₇H₂₉IN₆O₂ 596.1, observed LCMS m/z 597.1 (M+H).

### Part E:

By essentially the same procedure given in Preparative Example 1, Part D, compound 34 can be prepared. HPLC-MS t_{R}=1.82 min (UV_{254 nm}); mass calculated for formula C₂₈H₂₉N₇O₂ 495.2, observed LCMS m/z 496.2 (M+H).

### Part F:

To the compound 34 (46 mg) in a 20 mL vial, KOH (52 mg) and t-BuOH (0.5 mL) was added and the resulting mixture was stirred for 1 hour at 80° C. The reaction mixture was cooled to room temperature, diluted with EtOAc and washed with brine. After concentration, the residue was purified with Prep-LC and gave the product 35 (12 mg). HPLC-MS t_{R}=3.02 min (UV_{254 nm}); mass calculated for formula C28H₃₁N₇O₃ 513.3, observed LCMS m/z 514.3 (M+H).

### Part G:

To the compound 35 (12 mg) in AcCN (0.5 mL) was added 4N HCl in dioxane (1 mL). The reaction mixture was stirred for 1 hr and then concentrated. Lyophilization from H₂O/AcCN gave the product 36 as solid. HPLC-MS t_{R}=3.02 min (UV_{254 nm}); mass calculated for formula C23H₂₃N₇O 413.2, observed LCMS m/z 414.2 (M+H).

By essentially the same procedure given in Preparative Example 2, compounds 37-41 given in Column 2 of Table 1 can be prepared.

TABLE 2

| | | | MS | |
|---|---|---|---|---|
| | | Exact | m/z | HPLC |
| Example | Structure | mass | (M + H) | MS t_{R} |
| 37 | 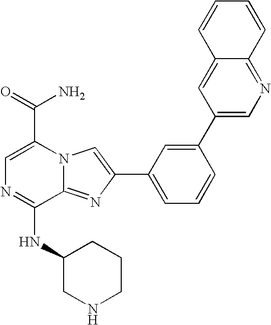 | 463.2 | 464.2 | 3.58 |
| 38 | 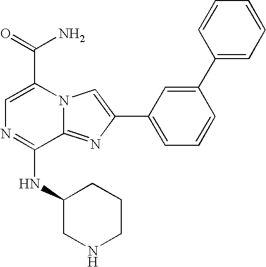 | 412.2 | 413.2 | 1.29 |
| 39 | 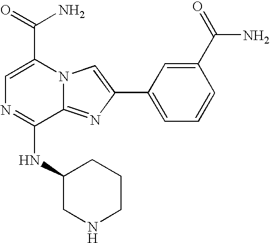 | 379.2 | 380.2 | 0.86 |
| 40 | 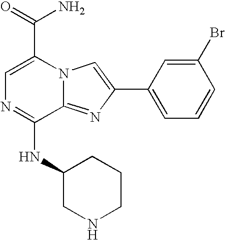 | 414.1 | 415.1 | 2.73 |
| 41 | 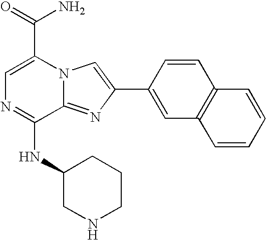 | 386.2 | 387.2 | 2.93 |

### Example 3

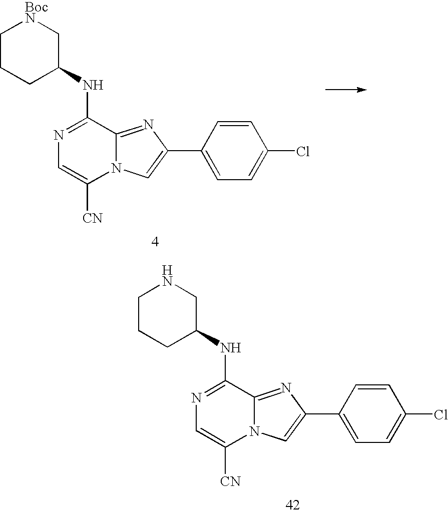

Deprotection of compound 4 (from Preparative Example 1) using the conditions described in Part G of Preparative Example 2 gave 42. HPLC-MS t_{R}=3.14 min (UV_{254 nm}); mass calculated for formula C₁₈H₁₇ClN₆ 352.1, observed LCMS m/z 353.1 (M+H).

By essentially the same procedure given in Preparative Example #, compounds 43-46 given in Column 2 of Table 3 can be prepared.

TABLE 3

| | | | MS | |
|---|---|---|---|---|
| | | Exact | m/z | HPLC |
| Example | Structure | mass | (M + H) | MS t_{R} |
| 43 | 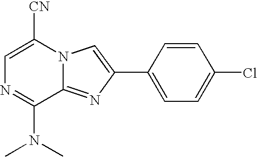 | 297.1 | 298.1 | 6.67 |
| 44 | 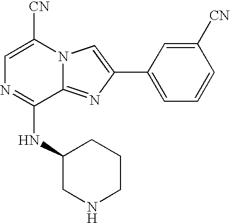 | 343.2 | 344.2 | 1.18 |
| 45 | 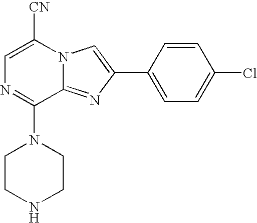 | 338.1 | 339.1 | 1.39 |
| 46 | 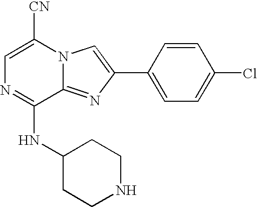 | 352.2 | 352.2 | 1.42 |

### Example 4

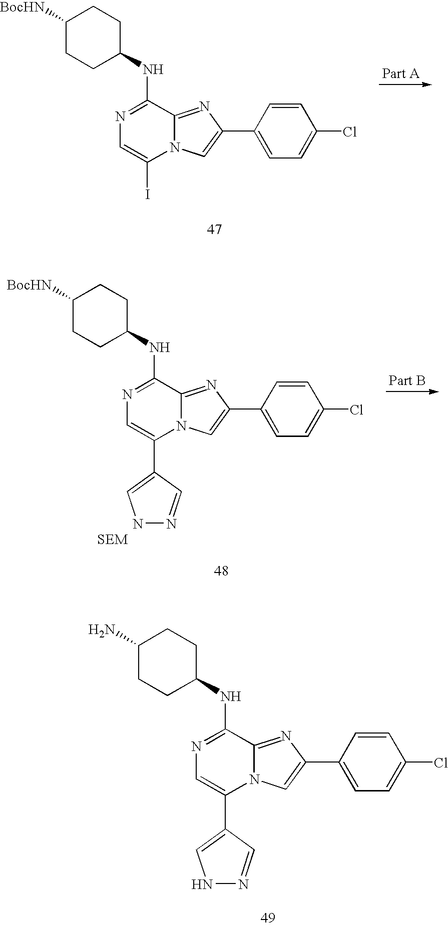

### Part A:

A sealed tube was charged with compound 47 (57 mg, 0.10 mmol), 1-methyl-4-(4,4,5,5-tetramethyl-1,3,2-dioxaborolan-2-yl)-1H-pyrazole (34 mg, 0.11 mmol), K₃PO₄—H₂O (49 mg, 0.21 mmol), and PdCl₂(dppf) (8.2 mg) 0.010 mmol). Dioxane (3 mL) and H₂O (0.3 mL) were added and the mixture was thoroughly degassed by bubbling Argon into the reaction mixture. This reaction was then heated at 75° C. overnight. After cooling to room temperature, it was diluted with EtOAc and washed with brine. After concentration the residue was purified with column (silica gel, EtOAc/hexane=25/75) to give the product 48 (32 mg) as oil. HPLC-MS t_{R}=2.48 min (UV_{254 nm}); mass calculated for formula 637.3, observed LCMS m/z 638.3 (M+H).

### Part B:

Deprotection of compound 48 using the conditions described in Part G of Example 2, Scheme 2 gave 49. HPLC-MS t_{R}=2.78 min (UV_{254 nm}); mass calculated for formula C21H₂₂ClN₇ 407.2, observed LCMS m/z 408.2 (M+H).

### Example 5

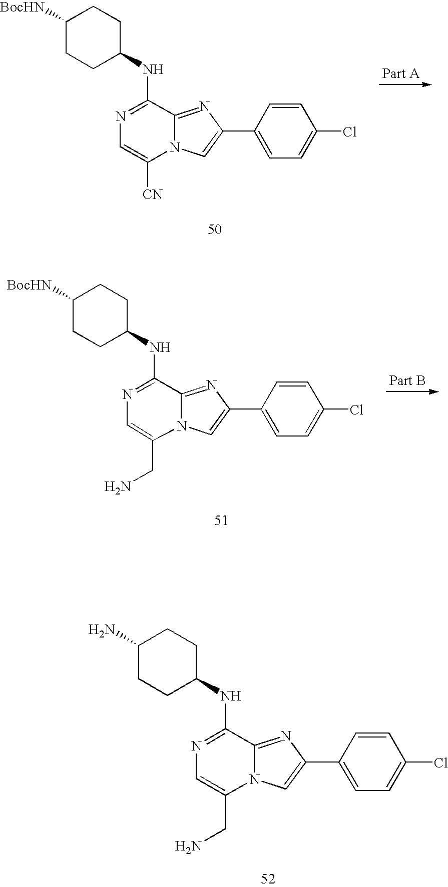

### Part A:

Compound 50 (5 mg, 0.01 mmol) in CH₂Cl₂ (1 mL) was treated with DIBAL (1 M, 0.15 mL) and stirred at room temperature for 5 min. The reaction mixture was then poured over a suspension on NaBH₄ in THF/MeOH (1:1) and then treated with NH₄Cl (sat. aq.) followed by NaOH (1 M). The mixture was extracted with CH₂Cl₂, dried over Na₂SO₄ and concentrated to give the compound 51 as oil. HPLC-MS t_{R}=1.52 min (UV_{254 nm}); mass calculated for formula C24H₃₁ClN₆O₂ 470.2, observed LCMS m/z 471.2 (M+H).

### Part B:

Deprotection of compound 51 using the conditions described in Part G of Example 2, Scheme 2 gave a solid which was purified with Prep-LC and gave the product 52. HPLC-MS t_{R}=2.20 min (UV_{254 nm}); mass calculated for formula C19H₂₃ClN₆ 370.2, observed LCMS m/z 371.2 (M+H).

### Biological Assay:

Table 4 shows the activity data for an illustrative representative list of compounds of the invention.

TABLE 4

| Compound No. | Mk2 IC50 = nM | CHK-1, IC50 = nM |
|---|---|---|
| 5 | 650 | 39 |
| 12 | 60 | 1083 |
| 39 | 810 | 55 |
| 44 | | 870 |

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A compound of the Formula: 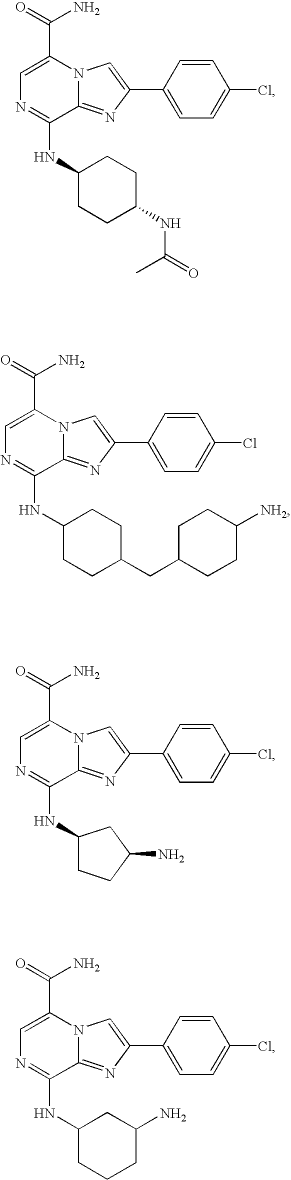 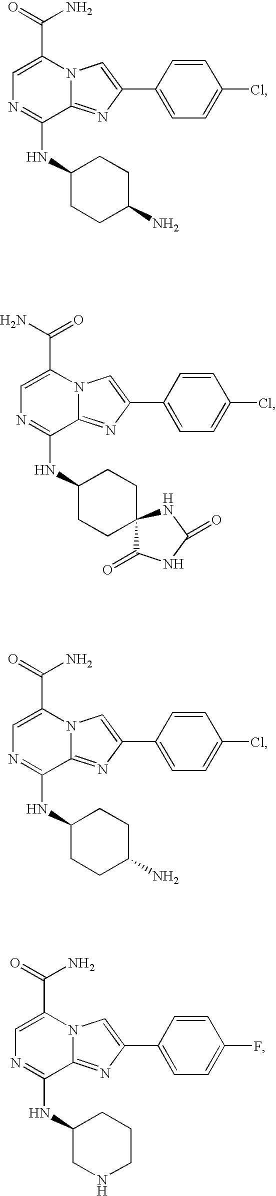 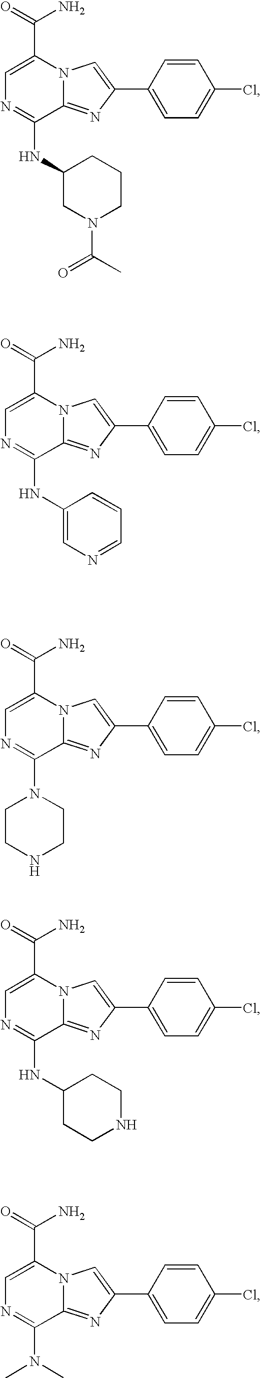 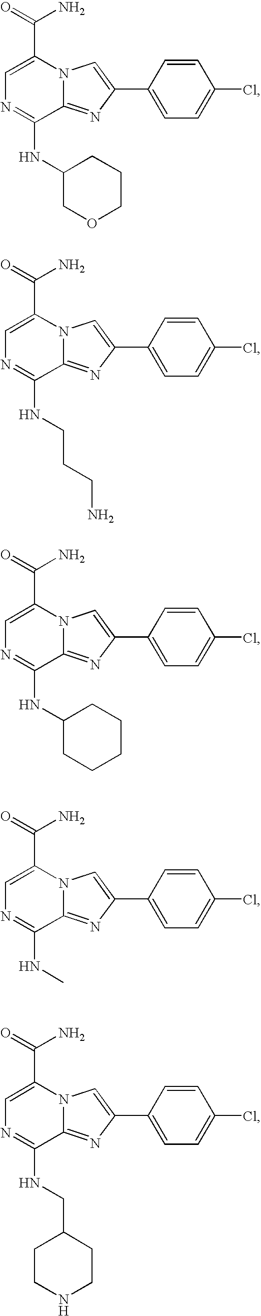 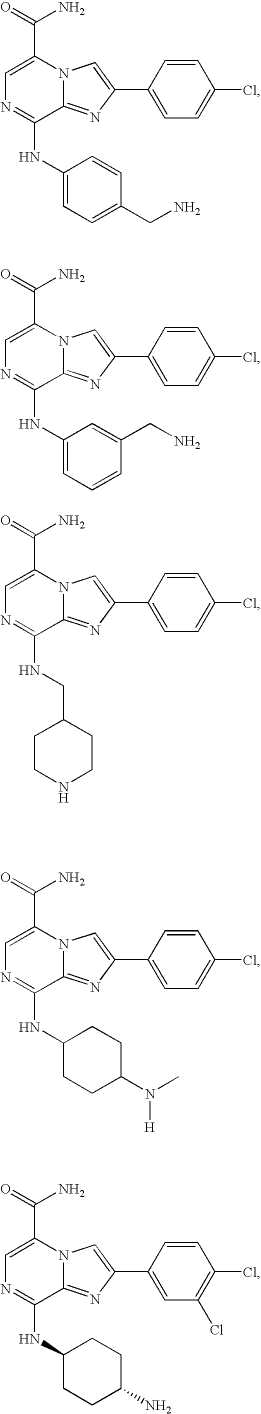 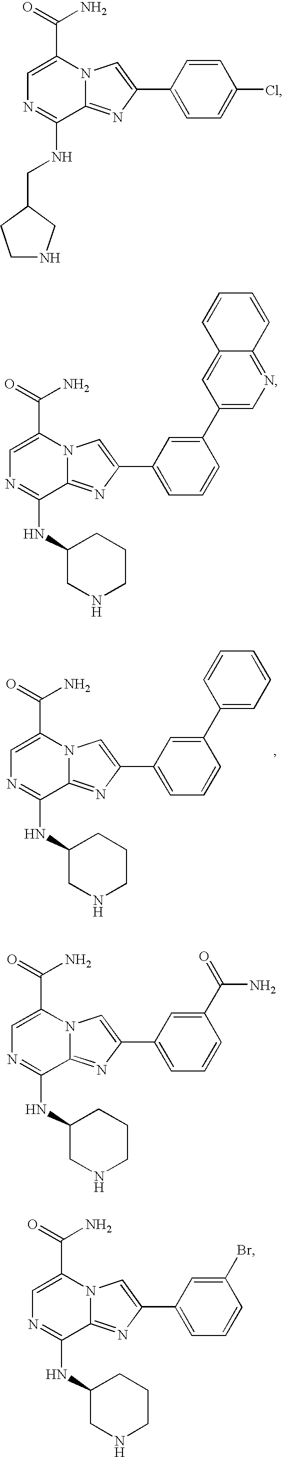 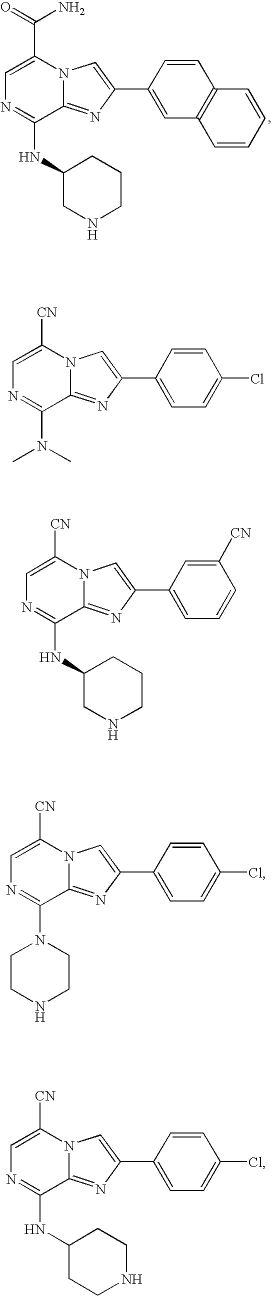 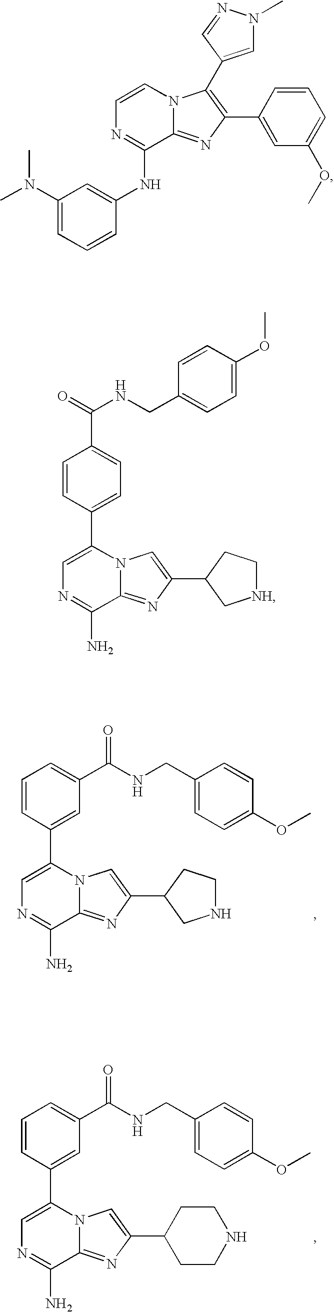 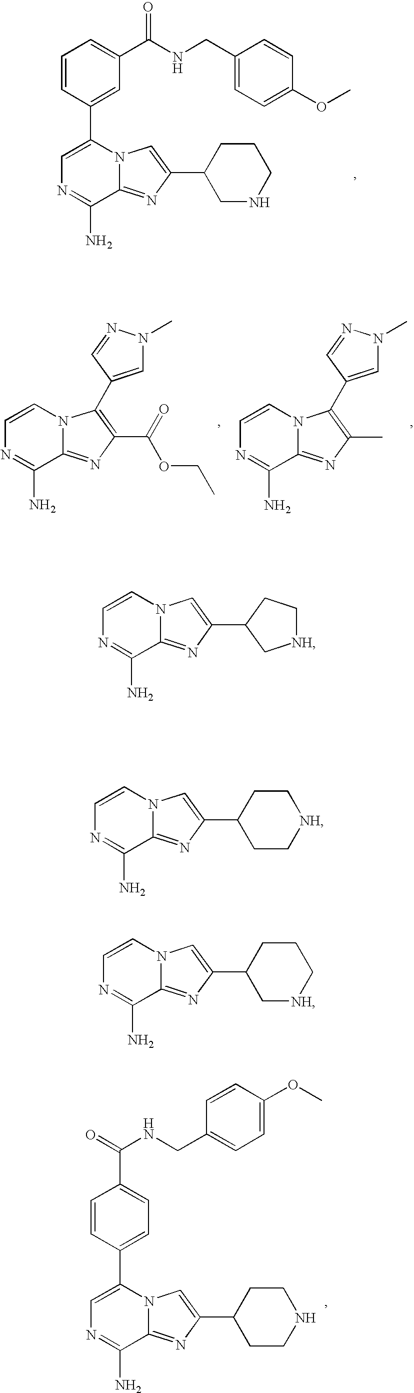 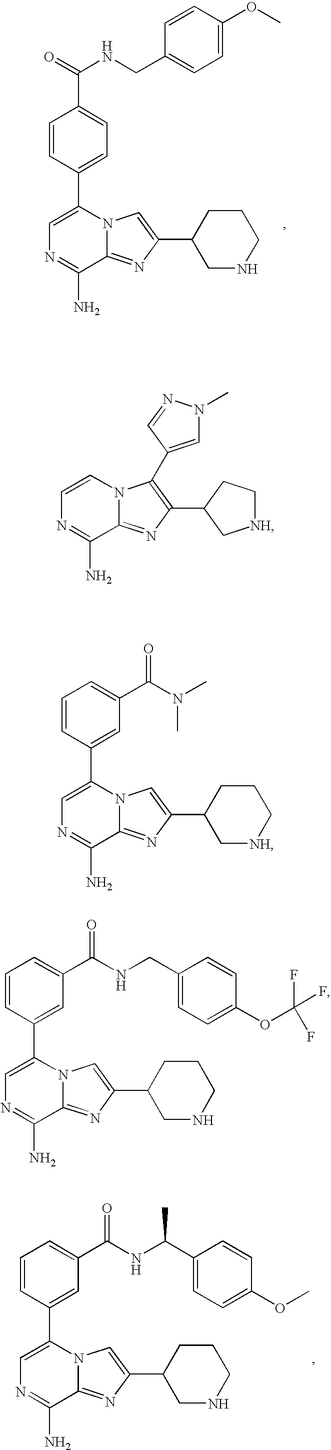 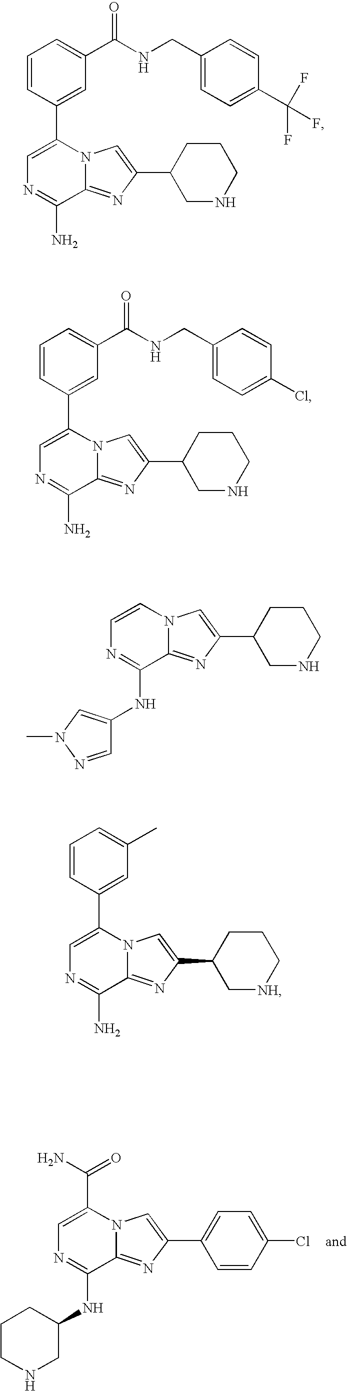 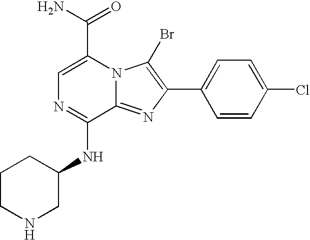
or a pharmaceutically acceptable salt, solvate or ester thereof.

2. A compound according to claim 1 or a pharmaceutically acceptable salt, solvate, ester or prodrug thereof in purified form.

3. A pharmaceutical composition comprising a therapeutically effective amount of at least one compound of claim 1 or a pharmaceutically acceptable salt, solvate, ester or prodrug thereof in combination with at least one pharmaceutically acceptable carrier.

4. The pharmaceutical composition according to claim 3, further comprising one or more anti-cancer agents different from the compound of claim 1.

5. The pharmaceutical composition according to claim 4, wherein the one or more anti-cancer agents are selected from the group consisting of cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGFR, Gleevec®, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux®, Liposomal, Thiotepa, Altretamine, Melphalan, Trastuzumab, Lerozole, Fulvestrant, Exemestane, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

6. The pharmaceutical composition according to claim 3, further comprising one or more anti-inflammatory agents different from the compound of claim 1.

7. The pharmaceutical composition according to claim 6, wherein the one or more anti-inflammatory agents are selected from the group consisting of disease modifying anti-rheumatic drugs (DMARDS), non-steroidal anti-inflammatory drugs (NSAIDs), cycloxygenase-2 selective (COX-2) inhibitors, COX-1 inhibitors, immunosuppressives, biological response modifiers (BRMs), anti-inflammatory agents and H1 antagonists.

8. A method of inhibiting one or more mitogen-activated protein kinase-activated protein kinase, or treating one or more diseases associated with a mitogen-activated protein kinase-activated protein kinase, comprising administering a therapeutically effective amount of at least one compound of claim 1 or a pharmaceutically acceptable salt, solvate, ester or prodrug thereof to a patient in need of such inhibition.

9. A method of treating one or more diseases associated with mitogen-activated protein kinase-activated protein kinase, comprising administering to a mammal in need of such treatment
an amount of a first compound, which is a compound of claim 1, or a pharmaceutically acceptable salt, solvate, ester or prodrug thereof and
an amount of at least one second compound, the second compound being an anti-cancer agent different from the compound of claim 1;
wherein the amounts of the first compound and the second compound result in a therapeutic effect.

10. A method of treating one or more diseases associated with mitogen-activated protein kinase-activated protein kinase, comprising administering to a mammal in need of such treatment
an amount of a first compound, which is a compound of claim 1, or a pharmaceutically acceptable salt, solvate, ester or prodrug thereof; and
an amount of at least one second compound, the second compound being an anti-inflammatory agent different from the compound of claim 1;
wherein the amounts of the first compound and the second compound result in a therapeutic effect.

11. The method according to claim 8, wherein the mitogen-activated protein kinase-activated protein kinase is MK2.

12. The method according to claim 8, wherein the mitogen-activated protein kinase-activated protein kinase is MK1.

13. The method according to claim 8, wherein the disease is selected from the group consisting of:
cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;
acute and, Chronic myelogenous leukemia, myelodysplastic syndrome and promyelocytic leukemia;
fibrosarcoma, rhabdomyosarcoma;
astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma
rheumatoid arthritis, osteoarthritis, periodontitis, gingivitis, corneal ulceration, solid tumor growth and tumor invasion by secondary metastases, neovascular glaucoma, inflammatory bowel disease, multiple sclerosis and psoriasis.

14. The method according to claim 8, further comprising radiation therapy.

15. The method according to claim 9, wherein the anti-cancer agent is selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGFR, Gleevec®, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux®, Liposomal, Thiotepa, Altretamine, Melphalan, Trastuzumab, Lerozole, Fulvestrant, Exemestane, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

16. The method according to claim 10, wherein the anti-inflammatory agent is selected from the group consisting of disease modifying anti-rheumatic drugs (DMARDS), non-steroidal anti-inflammatory drugs (NSAIDs), cycloxygenase-2 selective (COX-2) inhibitors, COX-1 inhibitors, immunosuppressives, biological response modifiers (BRMs), anti-inflammatory agents and H1 antagonists.

17. A method of inhibiting one or more Checkpoint kinases, or treating, or slowing the progression of, a disease associated with one or more Checkpoint kinases, in a patient in need thereof, comprising administering to the patient a therapeutically effective amount of at least one compound of claim 1 or a pharmaceutically acceptable salt, solvate, ester or prodrug thereof.

18. A method of treating one or more diseases associated with Checkpoint kinase, comprising administering to a mammal in need of such treatment
an amount of a first compound, which is a compound of claim 1, or a pharmaceutically acceptable salt, solvate, ester or prodrug thereof; and
an amount of at least one second compound, the second compound being an anti-cancer agent;
wherein the amounts of the first compound and the second compound result in a therapeutic effect.

19. The method of claim 18, wherein anti-cancer agent is selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGER, Gleevec®, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradioi, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin; Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux®, Liposomal, Thiotepa, Altretamine, Melphalan, Trastuzumab, Lerozole, Fulvestrant, Exemestane, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

20. A method of treating, or slowing the progression of, a disease associated with one or more Checkpoint kinases in a patient in need thereof, comprising administering a therapeutically effective amount of a pharmaceutical composition comprising in combination at least one pharmaceutically acceptable carrier and at least one compound according to claim 1, or a pharmaceutically acceptable salt, solvate, ester or prodrug thereof.

21. The method according to claim 17, wherein the Checkpoint kinase is Chk1.

22. The method according to claim 17, wherein the Checkpoint kinase is Chk2.

23. A method of inhibiting one or more tyrosine kinases, or treating, or slowing the progression of, a disease associated with one or more tyrosine kinases, in a patient in need thereof, comprising administering to the patient a therapeutically effective amount of at least one compound of claim 1 or a pharmaceutically acceptable salt, solvate, ester or prodrug thereof.

24. A method of treating one or more diseases associated with tyrosine kinase, comprising administering to a mammal in need of such treatment
an amount of a first compound, which is a compound of claim 1, or a pharmaceutically acceptable salt, solvate, ester or prodrug thereof; and
an amount of at least one second compound, the second compound being an anti-cancer agent;
wherein the amounts of the first compound and the second compound result in a therapeutic effect.

25. A method of treating, or slowing the progression of, a disease associated with one or more tyrosine kinases in a patient in need thereof, comprising administering a therapeutically effective amount of a pharmaceutical composition comprising in combination at least one pharmaceutically acceptable carrier and at least one compound according to claim 1 or a pharmaceutically acceptable salt, solvate, ester or prodrug thereof.

26. The method according to claim 23, wherein the tyrosine kinase is selected from the group consisting of VEGF-R2, EGFR, HER2, SRC, JAK and TEK.

27. The method according to claim 23, wherein the tyrosine kinase is VEGF-R2.

28. The method according to claim 23, wherein the tyrosine kinase is EGFR.

29. A method of inhibiting one or more Pim-1 kinases, or treating, or slowing the progression of, a disease associated with one or more Pim-1 kinases, in a patient in need thereof, comprising administering to the patient a therapeutically effective amount of at least one compound of claim 1 or a pharmaceutically acceptable salt, solvate, ester or prodrug thereof.

30. A method of treating one or more diseases associated with Pim-1 kinase, or treating, or slowing the progression of, a disease associated with one or more Pim-1 kinases, comprising administering to a mammal in need of such treatment an amount of a first compound, which is a compound of claim 1, or a pharmaceutically acceptable salt, solvate, ester or prodrug thereof; and an amount of at least one second compound, the second compound being an anti-cancer agent, wherein the amounts of the first compound and the second compound result in a therapeutic effect.

31. A method of treating a cancer comprising administering a therapeutically effective amount of at least one compound of claim 1, or a pharmaceutically acceptable salt, solvate, ester or prodrug thereof.

32. The method of claim 31, wherein said cancer is selected from the group consisting of: cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma and Burkett's lymphoma;
acute and chronic myelogenous leukemia, myelodysplastic syndrome and promyelocytic leukemia;
fibrosarcoma, rhabdomyosarcoma;
head and neck, mantle cell lymphoma, myeloma;
astrocytoma, neuroblastoma, glioma and schwannomas;
melanoma, seminoma; teratocarcinoma, osteosarcoma, xenoderoma pigmentosum, keratoctanthoma, thyroid follicular cancer and Kaposi's sarcoma.

33. A method of treating an inflammatory disease comprising administering a therapeutically effective amount of at least one compound of claim 1, or a pharmaceutically acceptable salt, solvate, ester or prodrug thereof.

34. The method of claim 33, wherein said inflammatory disease is selected from the group consisting of rheumatoid arthritis, osteoarthritis, periodontitis, gingivitis, corneal ulceration, solid tumor growth and tumor invasion by secondary metastases, neovascular glaucoma, inflammatory bowel disease, multiple sclerosis and psoriasis.

35. A method of treating a cancer, comprising administering to a mammal in need of such treatment
an amount of a first compound, which is a compound of claim 1, or a pharmaceutically acceptable salt, solvate, ester or prodrug thereof; and
an amount of at least one second compound, said second compound being an anti-cancer agent;
wherein the amounts of the first compound and said second compound result in a therapeutic effect.

36. The method of claim 35, further comprising radiation therapy.

37. The method of claim 35, wherein said anti-cancer agent is selected from the group consisting of cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGFR, Gleevec, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, Velcade, Zevalin, Trisenox, Xeloda, Vinorelbine, Porfimer, Erbitux®, Liposomal, Thiotepa, Altretamine, Melphalan, Trastuzumab, Lerozole, Fulvestrant, Exemestane, Fulvestrant, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

38. A method of treating an inflammatory disease, comprising administering to a mammal in need of such treatment
an amount of a first compound, which is a compound of claim 1, or a pharmaceutically acceptable salt, solvate, ester or prodrug thereof and
an amount of at least one second compound, said second compound being an anti-inflammatory agent;
wherein the amounts of the first compound and said second compound result in a therapeutic effect.

39. The method of claim 38, wherein said anti-inflammatory agent is selected from the group consisting of disease modifying anti-rheumatic drugs (DMARDS), non-steroidal anti-inflammatory drugs (NSAIDs), cycloxygenase-2 selective (COX-2) inhibitors, COX-1 inhibitors, immunosuppressives, biological response modifiers (BRMs), anti-inflammatory agents and H1 antagonists.

40. A method of inhibiting one or more cyclin dependent kinase, or treating one or more diseases associated with a cyclin dependent kinase, comprising administering a therapeutically effective amount of at least one compound of claim 1 or a pharmaceutically acceptable salt, solvate, ester or prodrug thereof to a patient in need of such inhibition.

41. A method of treating one or more diseases associated with cyclin dependent kinase, comprising administering to a mammal in need of such treatment
an amount of a first compound, which is a compound of claim 1, or a pharmaceutically acceptable salt, solvate, ester or prodrug thereof; and
an amount of at least one second compound, the second compound being an anti-cancer agent different from the compound of claim 1;
wherein the amounts of the first compound and the second compound result in a therapeutic effect.

42. The method according to claim 40, wherein the cyclin dependent kinase is CDK2.

43. The method according to claim 40, wherein the cyclin dependent kinase is CDK1.

44. The method according to claim 40, wherein the disease is selected from the group consisting of:
cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;
acute and chronic myelogenous leukemia, myelodysplastic syndrome and promyelocytic leukemia;
fibrosarcoma, rhabdomyosarcoma;
astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma.

45. The method according to claim 40, further comprising radiation therapy.

46. The method according to claim 41, wherein the anti-cancer agent is selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGFR, Gleevec®, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserein, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux®, Liposomal, Thiotepa, Altretamine, Melphalan, Trastuzumab, Lerozole, Fulvestrant, Exemestane, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

47. A method of inhibiting one or more mitogen-activated protein kinase-activated protein kinase, or treating one or more diseases associated with a mitogen-activated protein kinase-activated protein kinase, comprising administering a therapeutically effective amount of at least one compound of Formula I: 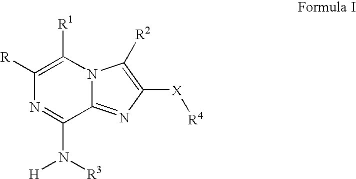
or a pharmaceutically acceptable salt, solvate, ester or prodrug thereof, wherein:
R is selected from the group consisting of H, halo, amino, alkyl, aryl, heteroaryl, heteroarylalkyl-, cycloalkyl, cycloalkylalkyl-, cycloalkenyl, cycloalkenylalkyl-, arylalkyl-, heterocyclyl, heterocyclylalkyl-, heterocyclenyl, heterocyclenylalkyl-, alkenyl-, alkynyl-, and —CN, wherein each of said alkyl, aryl, heteroaryl, heterocyclyl and cycloalkyl can be unsubstituted or optionally independently substituted with one or more moieties which can be the same or different each moiety being independently selected from halo, cyano, —N(R¹⁴R¹⁵), —C(O)—N(R¹⁴R¹⁵) and —N(R¹⁴)C(O)R¹⁵;
R¹ is selected from the group consisting of H, alkyl, aryl, arylalkyl-, heteroaryl, heteroarylalkyl-, cycloalkenyl, cycloalkenylalkyl, arylalkyl-, heterocyclyl, heterocyclylalkyl-, heterocyclenyl, heterocyclenylalkyl, and —C(O)—N(R¹⁴R¹⁵), wherein each of said alkyl, aryl, heteroaryl, heterocyclyl and cycloalkyl can be unsubstituted or optionally independently substituted with one or more moieties which can be the same or different each moiety being independently selected from halo, alkyl, aryl, cyano, —OR⁴, —SR¹⁴, —S(O)R, —S(O₂)R¹⁴—N(R¹⁴R¹⁵),
—C(O)O-alkyl, —C(O)—N(R¹⁴R¹⁵), —N(R¹⁴)S(O₂)R¹⁵ and —N(R¹⁴)C(O)R¹⁵;
R² is selected from the group consisting of H, halo, amino, alkyl, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵) and —N(R¹⁴)C(O)R¹⁵;
R³ is selected from the group consisting of H, alkyl, amino, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, —C(O)—N(R¹⁴R¹⁵) and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵) and —N(R¹⁴)C(O)R¹⁵;
X is selected from the group consisting of a covalent bond, —C(O)—NR¹⁴
—S(O₂)N(R¹⁴)—, —N(R¹⁴)—C(ON(R¹⁵), —C(O)—O—, —O—C(O)—, —O—C(O)—N(R¹⁴)—, —N(R¹⁴)—, —C(R¹⁴R¹⁵)—, —O—, —S—, —S(O)—, and —S(O₂)—,
or the moiety 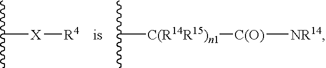
where n1 is 1-3;
R⁴ is selected from the group consisting of alkyl, —C(O)O-alkyl, heterocyclyl, aryl, and amino, wherein each of said alkyl, aryl and heterocyclyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵), —S(O₂)—N(R¹⁴R¹⁵), —S(O)—N(R¹⁴R¹⁵—), —N(R¹⁴)C(O)R¹⁵, —C(O)O-alkyl, —O(CO)O-alkyl, —C(O)O-aryl, —C(O)O-heteroaryl, —OR¹⁴, —SR¹⁴, —O—CO—N(R¹⁴R¹⁵)— and —N(R¹⁴)—CO—N(R¹⁴R¹⁵);
R⁶ is H, alkyl, cycloalkyl or aryl, wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OCF₃, —CF₃, dialkylamino-, alkylthio-, alkylsulfonyl-, dialkylaminosulfonyl-, —NO₂, —C(O)O-alkyl,
—O(CO)O-alkyl, —C(O)O-aryl, and —C(O)O-heteroaryl;
R⁷ is H, alkyl, cycloalkyl or aryl wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OF₃, —CF₃, dialkylamino-, alkylthio-, alkylsulfonyl-, dialkylaminosulfonyl-, —NO₂, —C(O)O-alkyl,
—O(CO)O-alkyl, —C(O)O-aryl, and —C(O)O-heteroaryl; and
R¹⁴ and R¹⁵ can be the same or different, each being independently selected from the group consisting of H, alkyl, aryl, heteroaryl, arylalkyl, cycloalkyl, heterocyclyl, acyl, halo, hydroxyalkyl, trifluoromethyl, —OR⁶,
—OCF₃, —N(R⁶R⁷), —SR⁶, —S(O₂)R⁶, —CN, —S(O₂)N(R⁶R⁷) and —NO₂, wherein each of said alkyl, aryl, heteroaryl, cycloalkyl and heterocyclyl can be unsubstituted or optionally independently substituted with one or more moieties which can be the same or different each moiety being independently selected from halo, alkyl, cyano, acyl, hydroxyalkyl., trifluoromethyl, —OR⁶, —OCF₃, —CF₃, —N(R⁶R⁷), —SR⁶, —S(O₂)R⁶,
—S(O₂)N(R⁶R⁷), —NO₂, —C(O)O-alkyl, —O(CO)O-alkyl, —C(O)O-aryl, and —C(O)O-heteroaryl;
with the proviso that the invention excludes the following compounds:

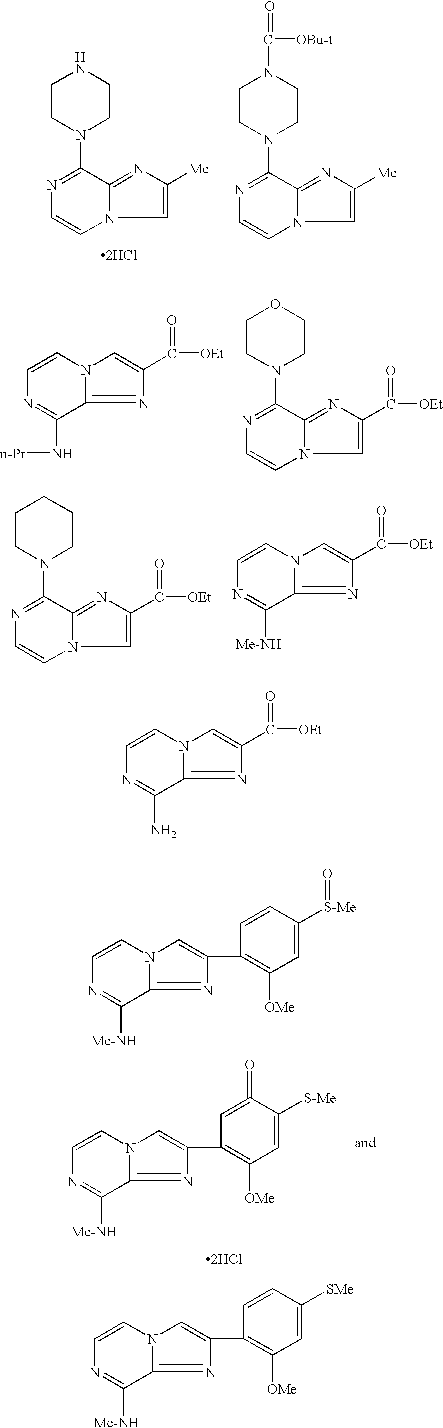

48. A method of treating one or more diseases associated with mitogen-activated protein kinase-activated protein kinase, comprising administering to a mammal in need of such treatment an amount of a first compound, which is a compound of Formula I: 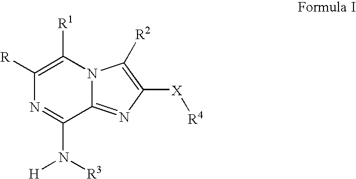
or a pharmaceutically acceptable salt, solvate, ester or prodrug thereof, wherein:
R is selected from the group consisting of H, halo, amino, alkyl, aryl, hetearyl, heteroarylalkyl-, cycloalkyl, cycloalkylalkyl-, cycloalkenyl, cycloalkenylalkyl-, arylalkyl-, heterocyclyl, heterocyclylalkyl-, heterocyclenyl, heterocyclenylalkyl-, alkenyl-, alkynyl-, and —CN, wherein each of said alkyl, aryl, heteroaryl, heterocyclyl and cycloalkyl can be unsubstituted or optionally independently substituted with one or more moieties which can be the same or different each moiety being independently selected from halo, cyano, —N(R¹⁴R¹⁵), —C(O)—N(R¹⁴R¹⁵) and —N(R¹⁴)C(O)R¹⁵;
R¹ is selected from the group consisting of H, alkyl, aryl, arylalkyl-, heteroaryl, heteroarylalkyl-, cycloalkenyl, cycloalkenylalkyl, arylalkyl-, heterocyclyl, heterocyclylalkyl-, heterocyclenyl, heterocyclenylalkyl, and —C(O)—N(R¹⁴R¹⁵), wherein each of said alkyl, aryl, heteroaryl, heterocyclyl and cycloalkyl can be unsubstituted or optionally independently substituted with one or more moieties which can be the same or different each moiety being independently selected from halo, alkyl, aryl, cyano, —OR¹⁴, —SR¹⁴, —S(O)R¹⁴, —S(O₂)R¹⁴—N(R¹⁴R¹⁵),
—C(O)O-alkyl, —C(O)—N(R¹⁴R¹⁵), —N(R¹⁴)S(O₂)R¹⁵, and —N(R¹⁴)C(O)R¹⁵;
R² is selected from the group consisting of H, halo, amino, alkyl, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O—N(R¹⁴R¹⁵) and —N(R¹⁴)C(O)R¹⁵,
R³ is selected from the group consisting of H, alkyl, amino, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, —C(O)—N(R¹⁴R¹⁵) and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵) and —N(R¹⁴)C(O)R¹⁵;
X is selected from the group consisting of a covalent bond, —C(O)—NR¹⁴—S(O₂)N(R¹⁴)—, —N(R¹⁴)—C(O)—N(R¹⁵), —C(O)—O—, —O—C(O)—, —O—C(O)—N(R¹⁴)—, —N(R^{14)—, —C(R}¹⁴R¹⁵)—, —O—, —S—, —S(O)—, and —S(O₂)—;
or the moiety 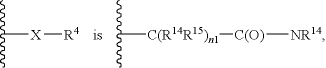
where n1 is 1-3;
R⁴ is selected from the group consisting of alkyl, —C(O)O-alkyl, heterocyclyl, aryl, and amino, wherein each of said alkyl, aryl and heterocyclyl can be unsubstituted or optionally independently substituted with one or more moieties which can be the same or different each moiety being independently selected from halo, alkyl, cyano, —N(R¹⁴R¹⁵),
—C(O)—N(R¹⁴R¹⁵), —S(O₂)—N(R¹⁴R¹⁵), —S(O)—N(R¹⁴R¹⁵), —N(R¹⁴)C(O)R¹⁵, —C(O)O-alkyl, —O(CO)O-alkyl, —C(O)O-aryl, —C(O)O-heteroaryl, —OR¹⁴—SR¹⁴—O—CO—N(R¹⁴R¹⁵)— and —N(R¹⁴)—CO—N(R¹⁴R¹⁵);
R⁶ is H, alkyl, cycloalkyl or aryl, wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OCF₃, —CF₃, dialkylamino-, alkylthio-, alkylsulfonyl-, dialkylaminosulfonyl-, —NO₂, —C(O)O-alkyl,
—O(CO)O-alkyl, —C(O)O-aryl, and —C(O)O-heteroaryl;
R⁷ is H, alkyl, cycloalkyl or aryl wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OCF₃, —CF₃, dialkylamino-, alkylthio-, alkylsulfonyl-, dialkylaminosulfonyl-, —NO₂, —C(O)O-alkyl,
—O(CO)O-alkyl, —C(O)O-aryl, and —CO(O)-heteroaryl; and
R¹⁴ and R¹⁵ can be the same or different, each being independently selected from the group consisting of H, alkyl, aryl, heteroaryl, arylalkyl, cycloalkyl, heterocyclyl, acyl, halo, hydroxyalkyl, trifluoromethyl, —OR⁶,
—OCF₃, —N(R⁶R⁷), —SR⁶, —S(O₂)R⁶, —CN, —S(O₂)N(R⁶R⁷) and —NO₂, wherein each of said alkyl, aryl, heteroaryl, cycloalkyl and heterocyclyl can be unsubstituted or optionally independently substituted with one or more moieties which can be the same or different each moiety being independently selected from halo, alkyl, cyano, acyl, hydroxyalkyl, trifluoromethyl, —OR⁶, —OCF₃, —CF₃, —N(R⁶R⁷), —SR⁶, —S(O₂)R⁶,
—S(O₂)N(R⁶R⁷), —NO₂, —C(O)O-alkyl, —O(CO)O-alkyl, —C(O)O-aryl, and —C(O)O-heteroaryl;
with the proviso that the invention excludes the following compounds: 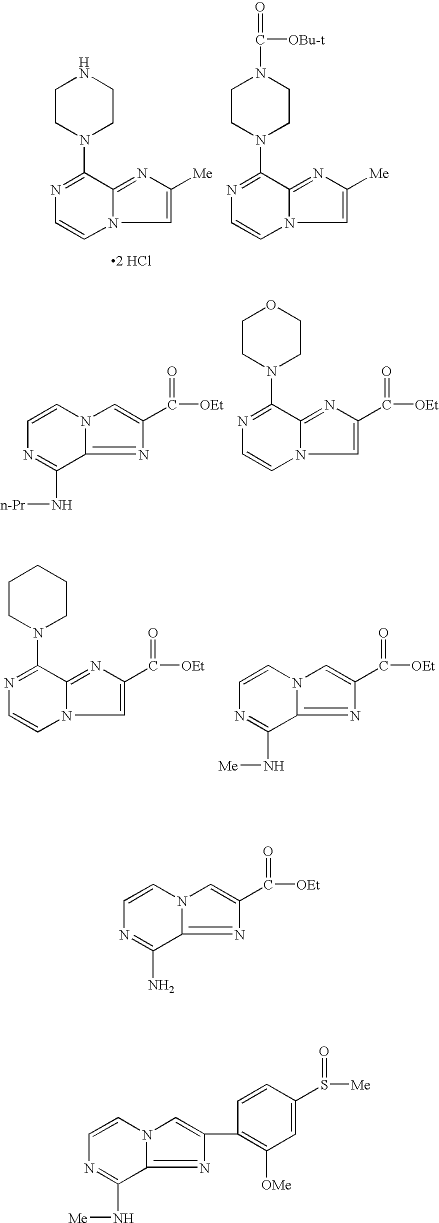 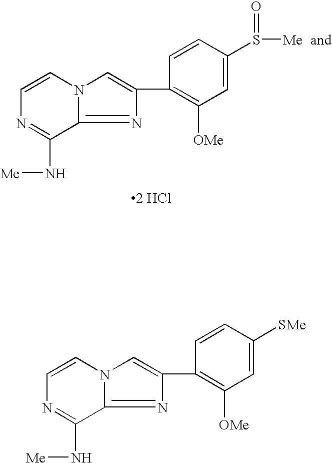
, or a pharmaceutically acceptable salt, solvate, ester or prodrug thereof; and
an amount of at least one second compound, the second compound being an anti-cancer agent different from the compound of Formula I;
wherein the amounts of the first compound and the second compound result in a therapeutic effect.

49. The method according to claim 47, wherein the mitogen-activated protein kinase-activated protein kinase is MK2.

50. The method according to claim 47, wherein the mitogen-activated protein kinase-activated protein kinase is MK1.

51. The method according to claim 47, wherein the disease is selected from the group consisting of:
cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma myeloma, and Burkett's lymphoma;
acute and chronic myelogenous leukemia, myelodysplastic syndrome and promyelocytic leukemia;
fibrosarcoma, rhabdomyosarcoma;
astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma.

52. The method according to claim 41, further comprising radiation therapy.

53. The method according to claim 48, wherein the anti-cancer agent is selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGFR, Gleevec® intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux®, Liposomal, Thiotepa, Altretamine, Melphalan, Trastuzumab, Lerozole, Fulvestrant, Exemestane, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

54. A method of treating one or more diseases associated with mitogen-activated protein kinase-activated protein kinase, comprising administering to a mammal in need of such treatment an amount of a first compound, which is a compound of Formula I: 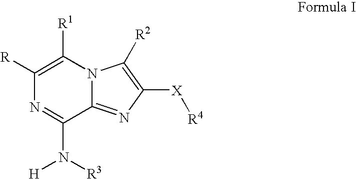
or a pharmaceutically acceptable salt, solvate, ester or prodrug thereof, wherein:
R is selected from the group consisting of H, halo, amino, alkyl, aryl, heteroaryl, heteroarylalkyl-, cycloalkyl, cycloalkylalkyl-, cycloalkenyl, cycloalkenylalkyl-, arylalkyl-, heterocyclyl, heterocyclylalkyl-, heterocyclenyl, heterocyclenylalkyl-, alkenyl-, alkynyl-, and —CN, wherein each of said alkyl, aryl, heteroaryl, heterocyclyl and cycloalkyl can be unsubstituted or optionally independently substituted with one or more moieties which can be the same or different each moiety being independently selected from halo, cyano, —N(R¹⁴R¹⁵), —C(O)—N(R¹⁴R¹⁵) and —N(R¹⁴)CO)R¹⁵;
R¹ is selected from the group consisting of H, alkyl, aryl, arylalkyl-, heteroaryl, heteroarylalkyl-, cycloalkenyl, cycloalkenylalkyl, arylalkyl-, heterocyclyl, heterocyclylalkyl-, heterocyclenyl, heterocyclenylalkyl, and —C(O)—N(R¹⁴R¹⁵), wherein each of said alkyl, aryl, heteroaryl, heterocyclyl and cycloalkyl can be unsubstituted or optionally independently substituted with one or more moieties which can be the same or different each moiety being independently selected from halo, alkyl, aryl, cyano, —OR¹⁴—SR¹⁴, —S(O)R¹⁴, —S(O₂)R¹⁴—N(R¹⁴R¹⁵),
—C(O)O-alkyl, —C(O—N(R¹⁴R¹⁵), —N(R¹⁴)S(O₂)R¹⁵, and
—N(R¹⁴)C(O)R¹⁵;
R² is selected from the group consisting of H, halo, amino, alkyl, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵) and —N(R¹⁴)C(O)R¹⁵;
R³ is selected from the group consisting of H, alkyl, amino, aryl, heteroaryl, heteroarylalkyl-, cycloalkyl, cycloalkylalkyl-, cycloalkenyl, cycloalkenylalkyl, arylalkyl-, heterocyclyl, heterocyclylalkyl-, heterocyclenyl, heterocyclenylalkyl, alkenyl, alkynyl, —C(O)—N(R¹⁴R¹⁵) and —CN, wherein each of said alkyl, aryl, heteroaryl, heterocyclyl and cycloalkyl can be unsubstituted or optionally independently substituted with one or more moieties which can be the same or different each moiety being independently selected from halo, alkyl, cyano, —N(R¹⁴R¹⁵), —C(O)—N(R¹⁴R¹⁵) and —N(R¹⁴)C(O)R¹⁵;
X is selected from the group consisting of a covalent bond, —C(O)—NR¹⁴, —S(O₂)N(R¹⁴)—, —N(R¹⁴)—C(O)—N(R¹⁵), —C(O)—O—, —O—C(O)—, —O—C(O)—N(R¹⁴)—N(R¹⁴)—, —C(R¹⁴R¹⁵)—, —O—, —S—, —S(O)—, and —S(O₂)—;
or the moiety 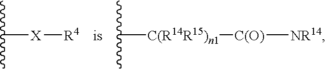
where n1 is 1-3;
R⁴ is selected from the group consisting of alkyl, —C(O)O-alkyl, heterocyclyl, aryl, and amino, wherein each of said alkyl, aryl and heterocyclyl can be unsubstituted or optionally independently substituted with one or more moieties which can be the same or different each moiety being independently selected from halo, alkyl, cyano, —N(R¹⁴R¹⁵),
—C(O)—N(R¹⁴R¹⁵), —S(O₂)—N(R¹⁴R¹⁵), —S(O)—N(R¹⁴R¹⁵), —N(R¹⁴)C(O)R¹⁵—C(O)O-alkyl, —OC(O)O-alkyl, —C(O)O-aryl, —C(O)O-heteroaryl, —OR¹⁴—SR¹⁴—O—CO—N(R¹⁴R¹⁵)— and —N(R¹⁴)—CO—N(R¹⁴R¹⁵);
R⁶ is H, alkyl, cycloalkyl or aryl, wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OCF₃, —CF₃, dialkylamino-, alkylthio-, alkylsulfonyl-, dialkylaminosulfonyl-, —NO₂, —C(O)O-alkyl,
—O(CO)O-alkyl, —C(O)O-aryl, and —C(O)O-heteroaryl;
R⁷ is H, alkyl, cycloalkyl or aryl wherein each of said alkyl, aryl and cycloalkyl can be unsubstituted or optionally independently substituted with one or more moieties which can be the same or different, each moiety being independently selected from halo, alkyl, cyano, acyl, hydroxyalkyl, trifluoromethyl, -alkoxy, —OCF₃, —CF₃, dialkylamino-, alkylthio-, alkylsulfonyl-, dialkylaminosulfonyl-, —NO₂, —C(O)O-alkyl,
—O(CO)O-alkyl, —C(O)O-aryl, and —C(O)O-heteroaryl; and
R¹⁴ and R¹⁵ can be the same or different, each being independently selected from the group consisting of H, alkyl, aryl, heteroaryl, arylalkyl, cycloalkyl, heterocyclyl, acyl, halo, hydroxyalkyl, trifluoromethyl, —OR⁶,
—OCF₃, —N(R⁶R⁷), —SR⁶, —S(O₂)R⁶, —CN, —S(O₂)N(R⁶R⁷) and —NO₂₁ wherein each of said alkyl, aryl, heteroaryl, cycloalkyl and heterocyclyl can be unsubstituted or optionally independently substituted with one or more moieties which can be the same or different each moiety being independently selected from halo, alkyl, cyano, acyl, hydroxyalkyl, trifluoromethyl, —OR⁶, —OCF₃, —CF₃, —N(R⁶R⁷), —SR⁶, —S(O₂)R⁶,
—S(O₂)N(R⁶R⁷), —NO₂, —C(O)O-alkyl, —O(CO)O-alkyl, —C(O)O-aryl, and —C(O)O-heteroaryl;
with the proviso that the invention excludes the following compounds: 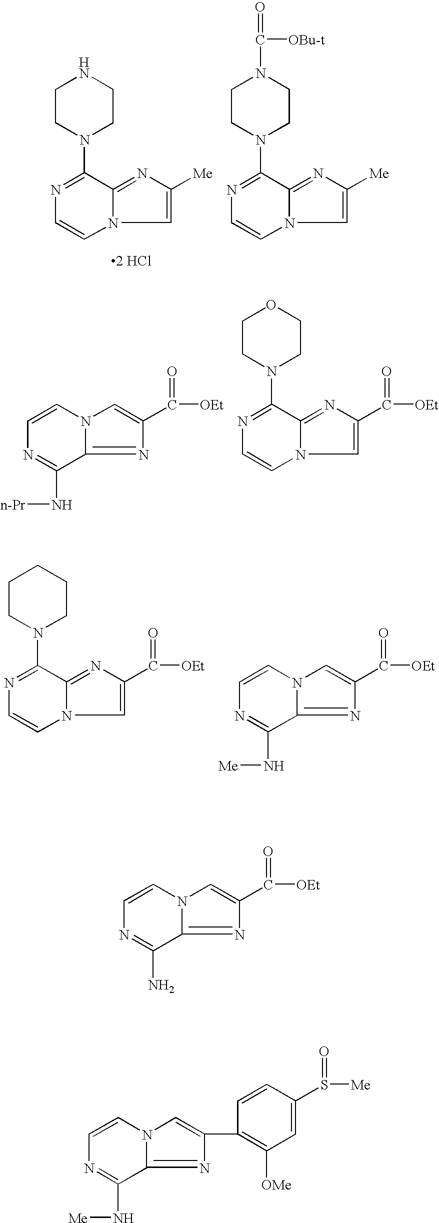 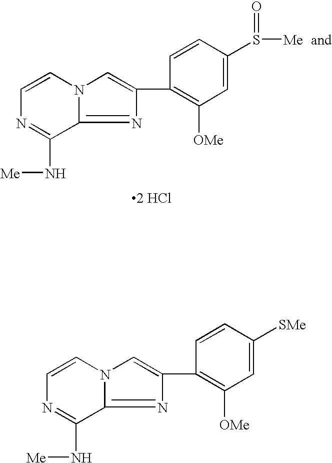
, or a pharmaceutically acceptable salt, solvate, ester or prodrug thereof; and
an amount of at least one second compound, the second compound being an anti-inflammatory agent different from the compound of Formula I;
wherein the amounts of the first compound and the second compound result in a therapeutic effect.

55. The method according to claim 47, wherein the disease is selected from the group consisting of: rheumatoid arthritis, osteoarthritis, periodontitis, gingivitis, corneal ulceration, solid tumor growth and tumor invasion by secondary metastases, neovascular glaucoma, inflammatory bowel disease, multiple sclerosis and psoriasis.

56. The method according to claim 47, wherein the anti-inflammatory agent is selected from the group consisting of disease modifying anti-rheumatic drugs (DMARDS), non-steroidal anti-inflammatory drugs (NSAIDs), cycloxygenase-2 selective (COX-2) inhibitors, COX-1 inhibitors, immunosuppressives, biological response modifiers (BRMs), anti-inflammatory agents and H1 antagonists.

57. A method of inhibiting one or more mitogen-activated protein kinase-activated protein kinase, or treating one or more diseases associated with a mitogen-activated protein kinase-activated protein kinase, comprising administering a therapeutically effective amount of at least one compound of Formula II: 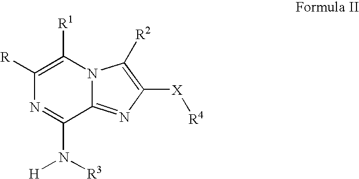
or a pharmaceutically acceptable salt, solvate, ester or prodrug thereof wherein:
R is H;
R¹ is CN or —C(O)—N(R¹⁴R¹⁵);
R² is H;
R³ is selected from the group consisting of alkyl, aryl, heteroaryl, cycloalkyl, cycloalkylalkyl-, heterocyclyl, and heterocyclylalkyl, wherein each of said alkyl, aryl, heteroaryl, cycloalkyl, cycloalkylalkyl-, heterocyclyl, and heterocyclylalkyl can be unsubstituted or optionally independently substituted with one or more moieties which can be the same or different each moiety being independently selected from, —N(R¹⁴R¹⁵), —C(O)—N(R¹⁴R¹⁵), —N(R¹⁴)C(O)R¹⁵ and spiroheterocyclyl group;
X is a covalent bond;
R⁴ is aryl, wherein said aryl can be unsubstituted or optionally independently substituted with one or more moieties which can be the same or different each moiety being independently selected from halo, cyano, —C(O)—N(R¹⁴R¹⁵), aryl, and heteroaryl;
R⁵ is H or alkyl or R⁴ and R⁵ together with the N to which each is attached form a heterocyclyl;
R¹⁴ and R¹⁵ can be the same or different, each being H or alkyl.

58. A method of treating one or more diseases associated with mitogen-activated protein kinase-activated protein kinase, comprising administering to a mammal in need of such treatment an amount of a first compound, which is a compound of Formula II: 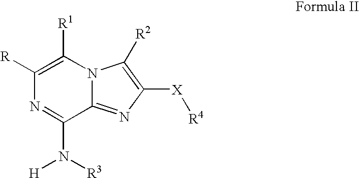
or a pharmaceutically acceptable salt, solvate, ester or prodrug thereof wherein:
R is H;
R¹ is CN or —C(O)—N(R¹⁴R¹⁵);
R² is H;
R³ is selected from the group consisting of alkyl, aryl, heteroaryl, cycloalkyl, cycloalkylalkyl-, heterocyclyl, and heterocyclylalkyl, wherein each of said alkyl, aryl, heteroaryl, cycloalkyl, cycloalkylalkyl-, heterocyclyl, and heterocyclylalkyl can be unsubstituted or optionally independently substituted with one or more moieties which can be the same or different each moiety being independently selected from, —N(R¹⁴R¹⁵), —C(O)—N(R¹⁴R¹⁵), —N(R¹⁴)C(O)R¹⁵ and spiroheterocyclyl group;
X is a covalent bond;
R⁴ is aryl, wherein said aryl can be unsubstituted or optionally independently substituted with one or more moieties which can be the same or different each moiety being independently selected from halo, cyano, —C(O)—N(R¹⁴R¹⁵), aryl, and heteroaryl;
R⁵ is H or alkyl or R⁴ and R⁵ together with the N to which each is attached form a heterocyclyl;
R¹⁴ and R¹⁵ can be the same or different, each being H or alkyl, or a
pharmaceutically acceptable salt, solvate, ester or prodrug thereof; and
an amount of at least one second compound, the second compound being an anti-cancer agent different from the compound of Formula II;
wherein the amounts of the first compound and the second compound result in a therapeutic effect.

59. The method according to claim 57, wherein the mitogen-activated protein kinase-activated protein kinase is MK2.

60. The method according to claim 57, wherein the mitogen-activated protein kinase-activated protein kinase is MK11.

61. The method according to claim 57, wherein the disease is selected from the group consisting of:
cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;
acute and chronic myelogenous leukemia, myelodysplastic syndrome and promyelocytic leukemia;
fibrosarcoma, rhabdomyosarcoma;
astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma.

62. The method according to claim 57, further comprising radiation therapy.

63. The method according to claim 58, wherein the anti-cancer agent is selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGFR, Gleevec® intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin. Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux®, Liposomal, Thiotepa, Altretamine, Melphalan, Trastuzumab, Lerozole, Fulvestrant, Exemestane, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

64. The method according to claim 57, wherein the disease is selected from the group consisting of, rheumatoid arthritis, osteoarthritis, periodontitis, gingivitis, corneal ulceration, solid tumor growth and tumor invasion by secondary metastases, neovascular glaucoma, inflammatory bowel disease, multiple sclerosis and psoriasis.

65. A method of treating one or more diseases associated with mitogen-activated protein kinase-activated protein kinase, comprising administering to a mammal in need of such treatment an amount of a first compound, which is a compound of Formula II: 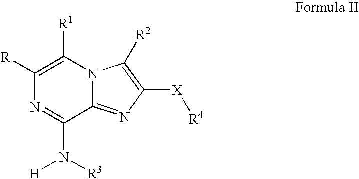
or a pharmaceutically acceptable salt, solvate, ester or prodrug thereof, wherein:
R is H;
R¹ is CN or —C(O)—N(R¹⁴R¹⁵);
R² is H;
R³ is selected from the group consisting of alkyl, aryl, heteroaryl, cycloalkyl, cycloalkylalkyl-, heterocyclyl, and heterocyclylalkyl, wherein each of said alkyl, aryl, heteroaryl, cycloalkyl, cycloalkylalkyl-, heterocyclyl, and heterocyclylalkyl can be unsubstituted or optionally independently substituted with one or more moieties which can be the same or different each moiety being independently selected from, —N(R¹⁴R¹⁵), —C(O)—N(R¹⁴R¹⁵), —N(R¹⁴)C(O)R⁵ and spiroheterocyclyl group;
X is a covalent bond;
R⁴ is aryl, wherein said aryl can be unsubstituted or optionally independently substituted with one or more moieties which can be the same or different each moiety being independently selected from halo, cyano, —C(O)—N(R¹⁴R¹⁵), aryl, and heteroaryl;
R⁵ is H or alkyl or R⁴ and R⁵ together with the N to which each is attached form a heterocyclyl;
R¹⁴ and R¹⁵ can be the same or different, each being H or alkyl, or a
pharmaceutically acceptable salt, solvate, ester or prodrug thereof; and
an amount of at least one second compound, the second compound being an anti-inflammatory agent different from the compound of Formula II;
wherein the amounts of the first compound and the second compound result in a therapeutic effect.

66. The method according to claim 55, wherein the anti-inflammatory agent is selected from the group consisting of disease modifying anti-rheumatic drugs (DMARDS), non-steroidal anti-inflammatory drugs (NSAIDs), cycloxygenase-2 selective (COX-2) inhibitors, COX-1 inhibitors, immunosuppressives, biological response modifiers (BRMs), anti-inflammatory agents and H1 antagonists.

67. A method of inhibiting one or more mitogen-activated protein kinase-activated protein kinase, or treating one or more diseases associated with a mitogen-activated protein kinase-activated protein kinase, comprising administering a therapeutically effective amount of at least one compound selected from the group consisting of:

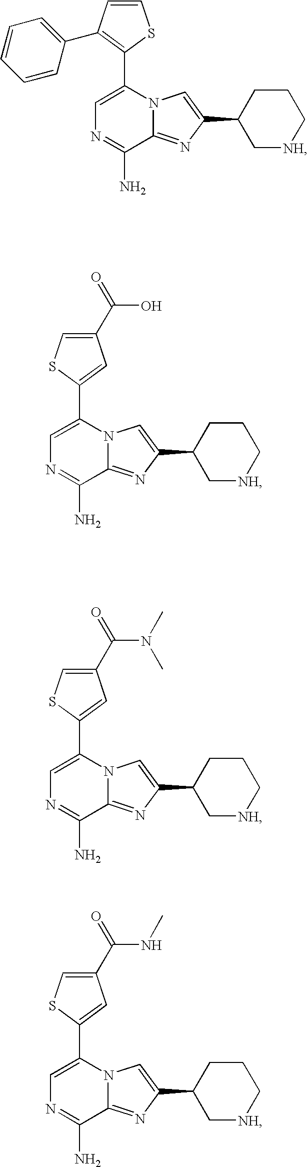

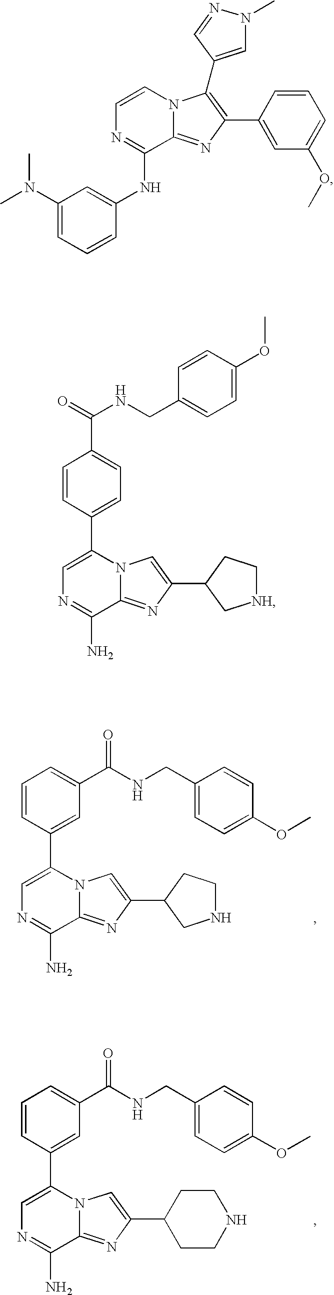

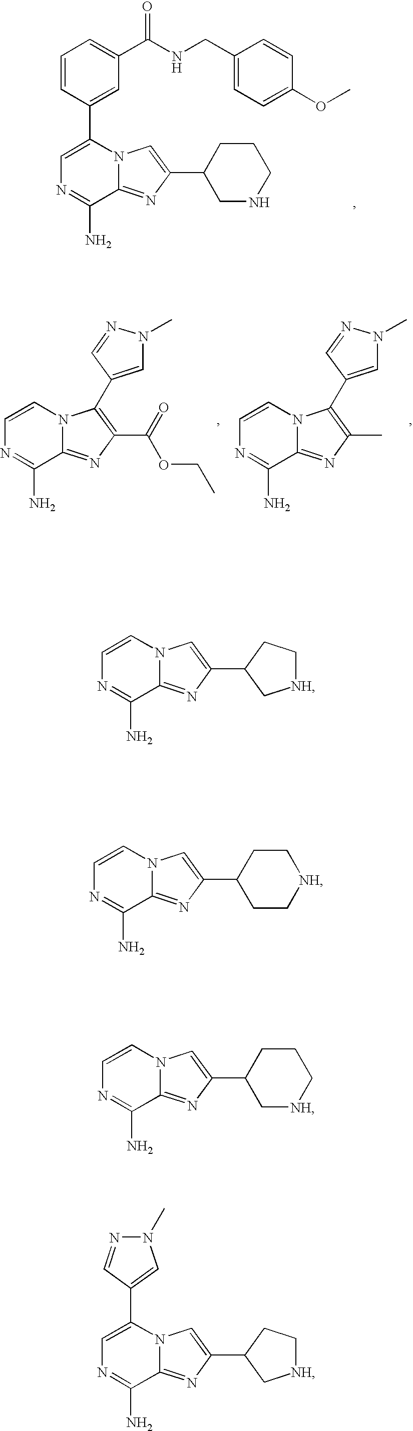

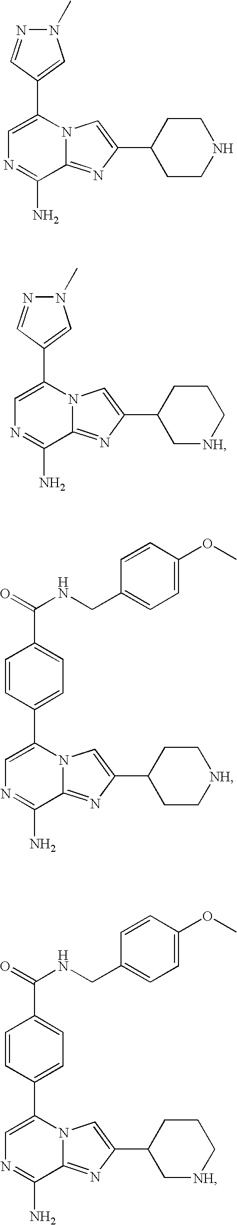

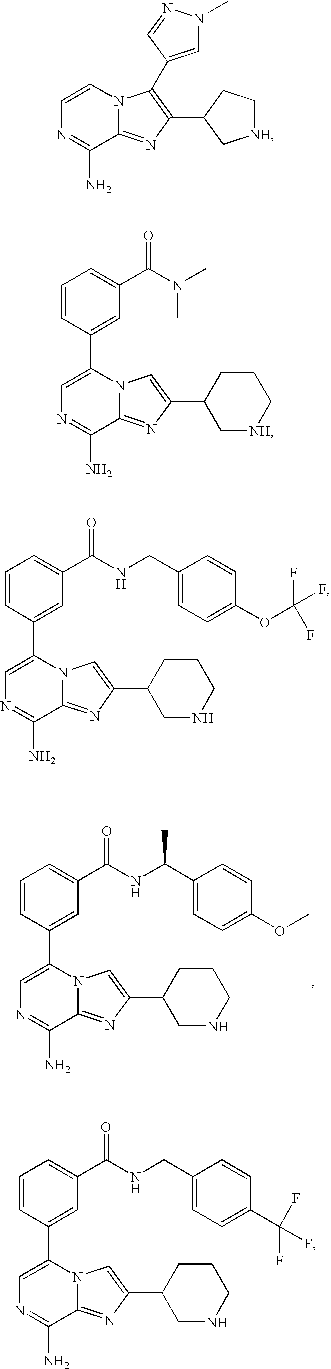

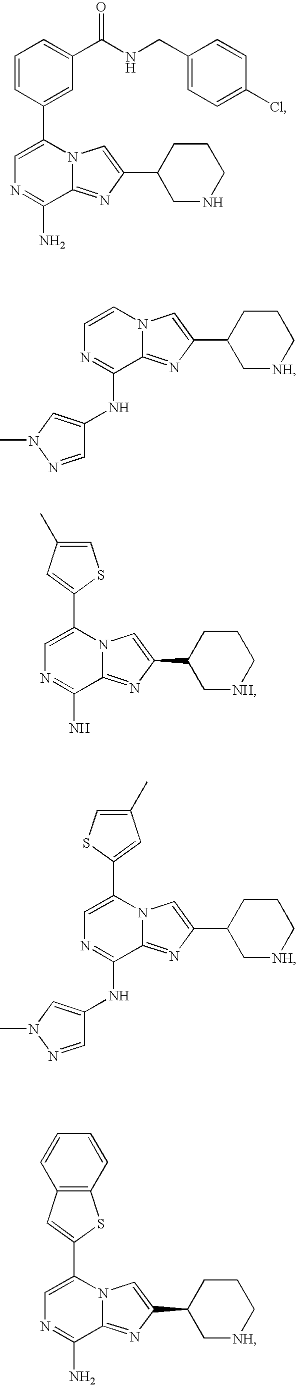

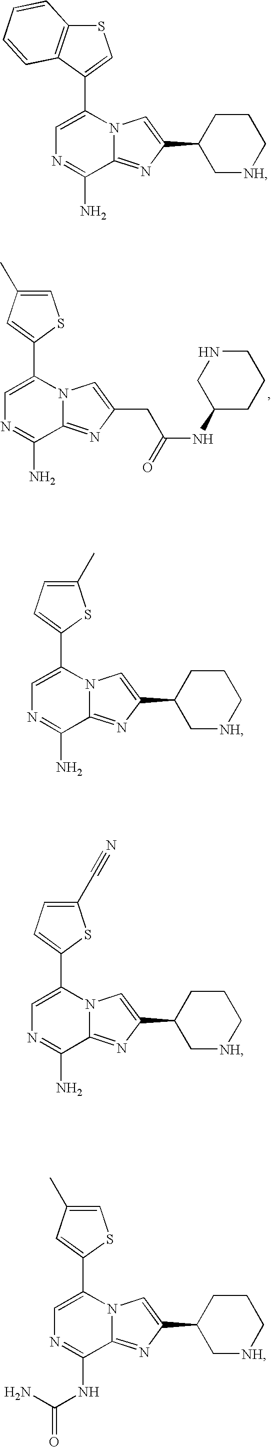

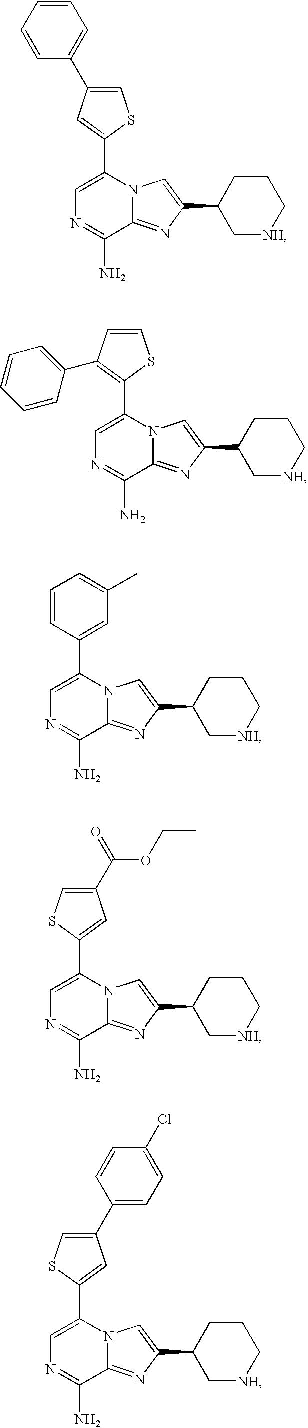

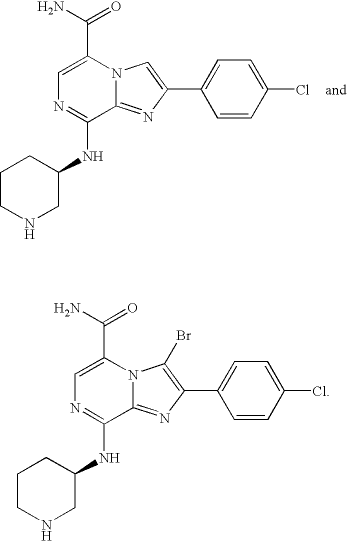

68. A method of treating one or more diseases associated with mitogen-activated protein kinase-activated protein kinase, comprising administering to a mammal in need of such treatment an amount of a first compound, which is a compound selected from the group consisting of: 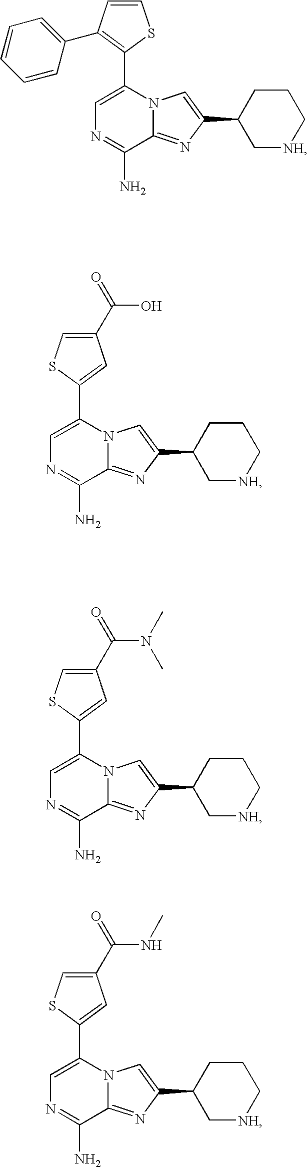 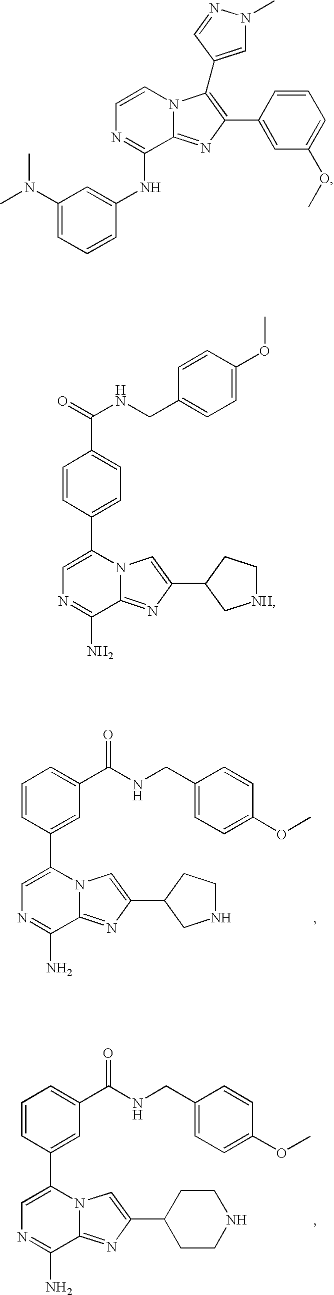 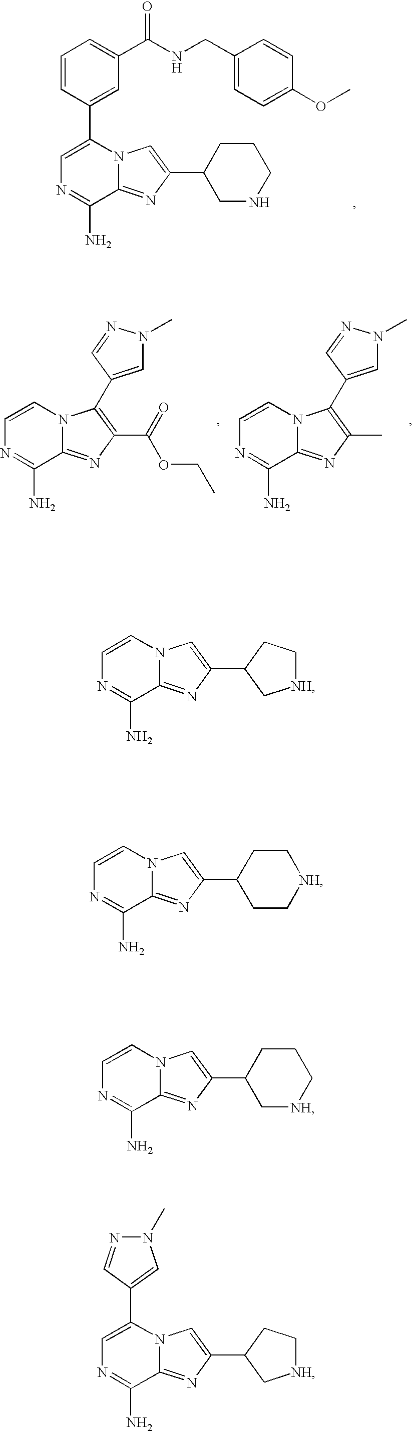 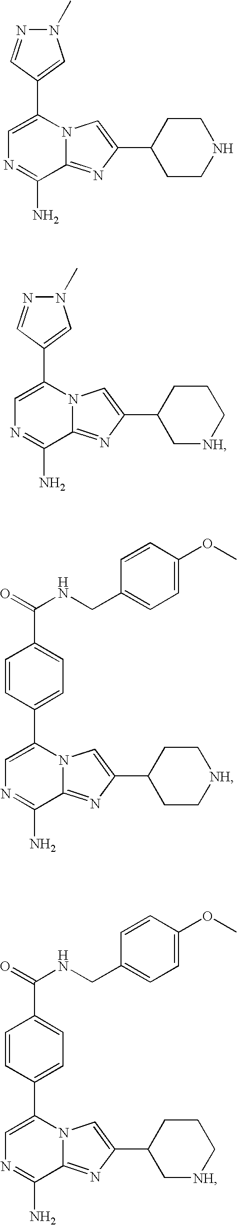 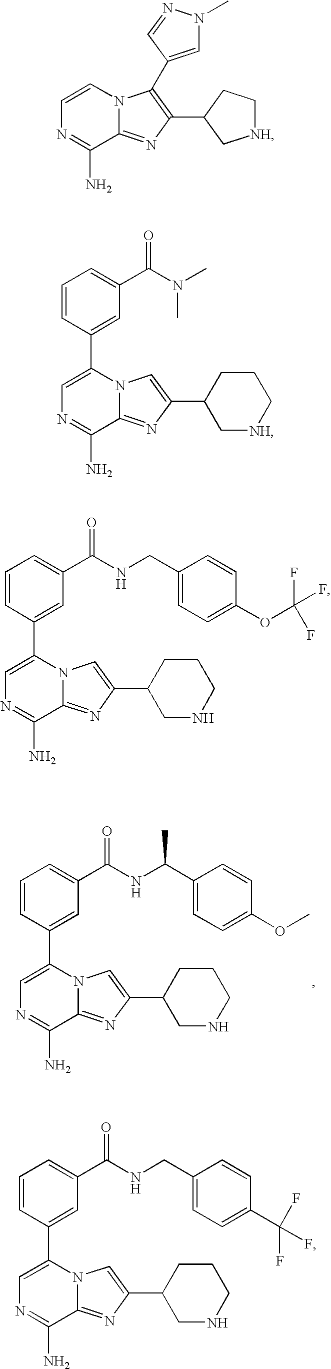 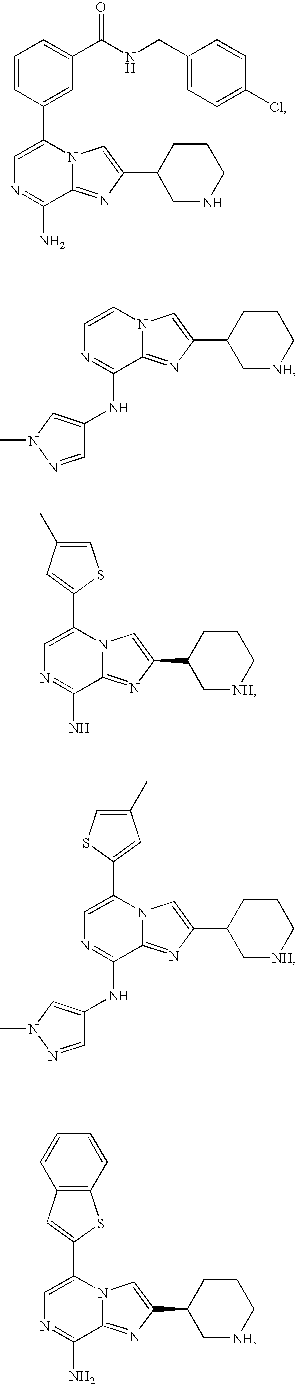 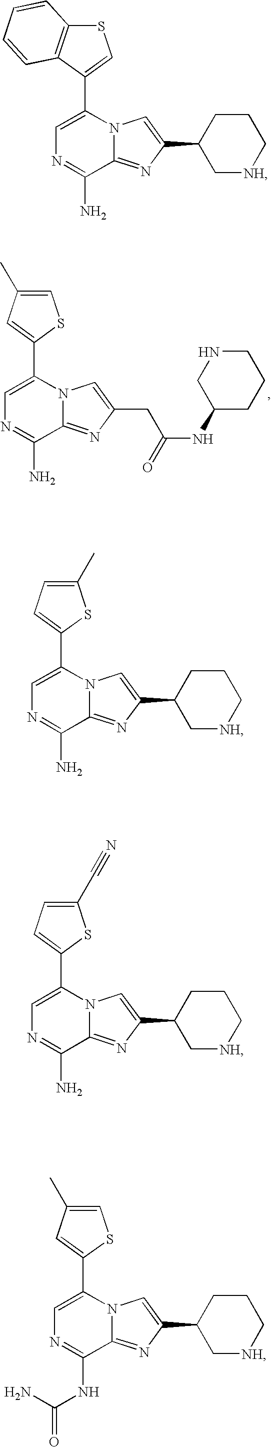 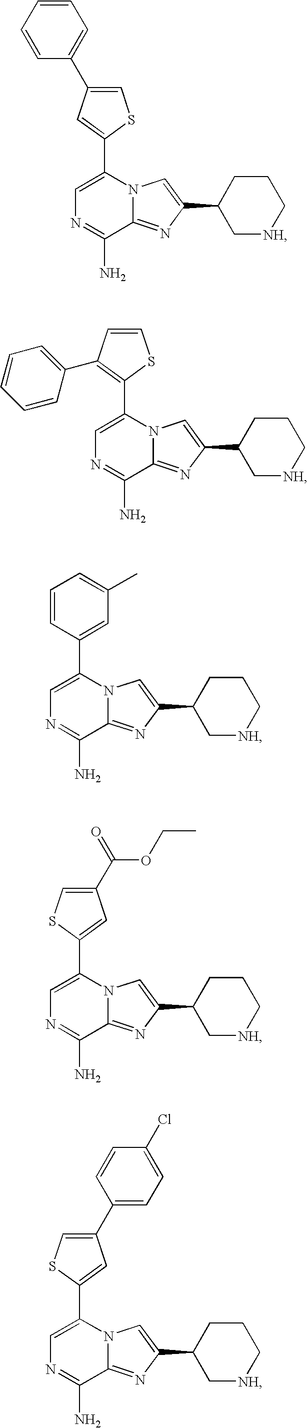 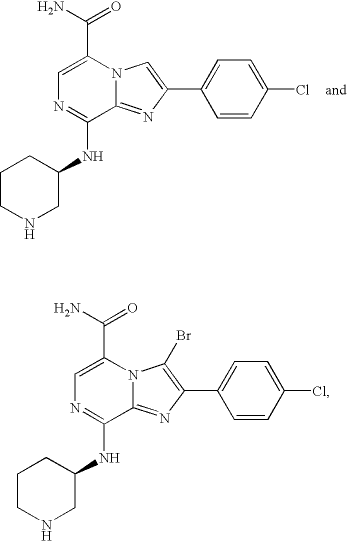
or a pharmaceutically acceptable salt, solvate, ester or prodrug thereof; and
an amount of at least one second compound, the second compound being an anti-cancer agent different from the first compound;
wherein the amounts of the first compound and the second compound result in a therapeutic effect.

69. The method according to claim 67, wherein the mitogen-activated protein kinase-activated protein kinase is MK2.

70. The method according to claim 67, wherein the mitogen-activated protein kinase-activated protein kinase is MK1.

71. The method according to claim 67, wherein the disease is selected from the group consisting of:
cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;
acute and chronic myelogenous leukemia, myelodysplastic syndrome and promyelocytic leukemia;
fibrosarcoma, rhabdomyosarcoma;
astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma.

72. The method according to claim 67, further comprising radiation therapy.

73. The method according to claim 68, wherein the anti-cancer agent is selected from the group consisting of a cytostatic agent, cisplatin, doxorubicin, taxotere, taxol, etoposide, rinotecan, camptostar, topotecan, paclitaxel, docetaxel, epothilones, tamoxifen, 5-fluorouracil, methoxtrexate, temozolomide, cyclophosphamide, SCH 66336, R115777, L778123, BMS 214662, Iressa®, Tarceva®, antibodies to EGFR, Gleevec®, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux®, Liposomal, Thiotepa, Altretamine, Melphalan, Trastuzumab, Lerozole, Fulvestrant, Exemestane, Ifosfomide, Rituximab, C225, Doxil, Ontak, Deposyt, Mylotarg, Campath, Celebrex, Sutent, Aranesp, Neupogen, Neulasta, Kepivance, SU11248, and PTK787.

74. The method according to claim 67, wherein the disease is selected from the group consisting of rheumatoid arthritis, osteoarthritis, periodontitis, gingivitis, corneal ulceration, solid tumor growth and tumor invasion by secondary metastases, neovascular glaucoma, inflammatory bowel disease, multiple sclerosis and psoriasis.

75. A method of treating one or more diseases associated with mitogen-activated protein kinase-activated protein kinase, comprising administering to a mammal in need of such treatment an amount of a first compound, which is a compound selected from the group consisting of: 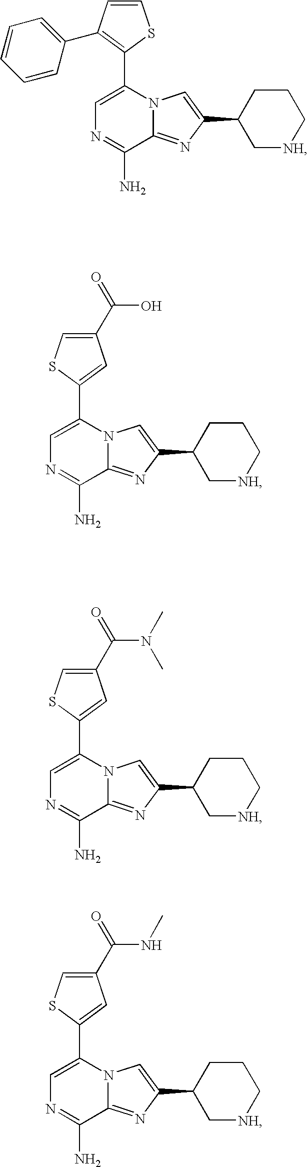 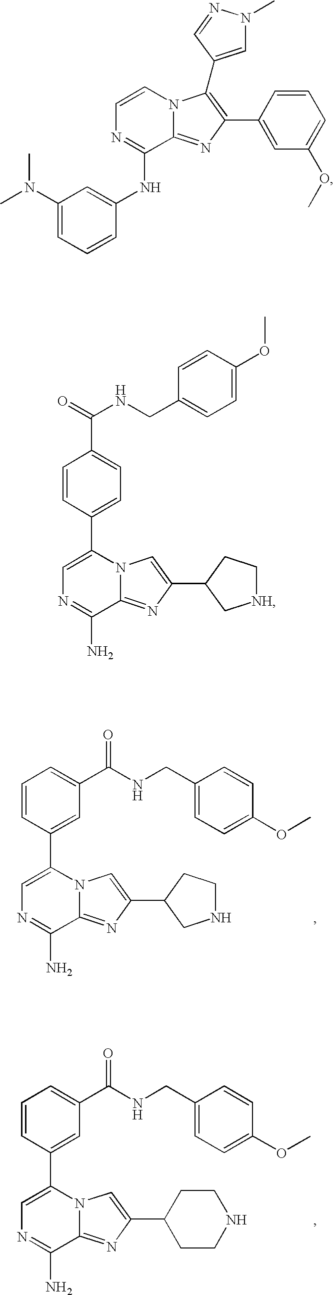 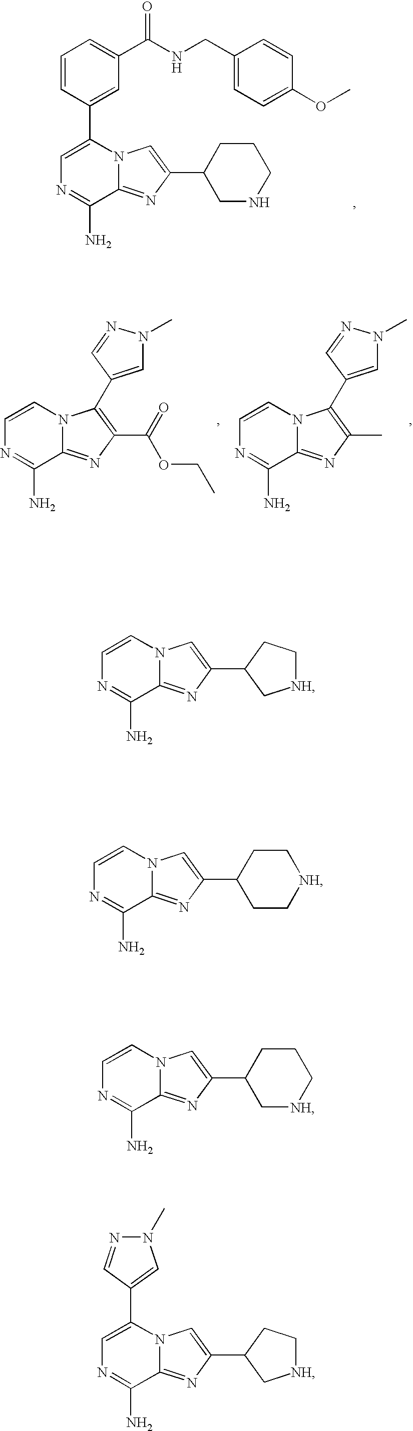 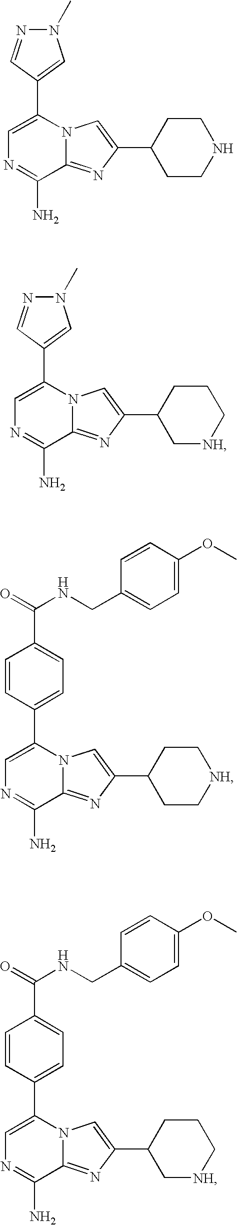 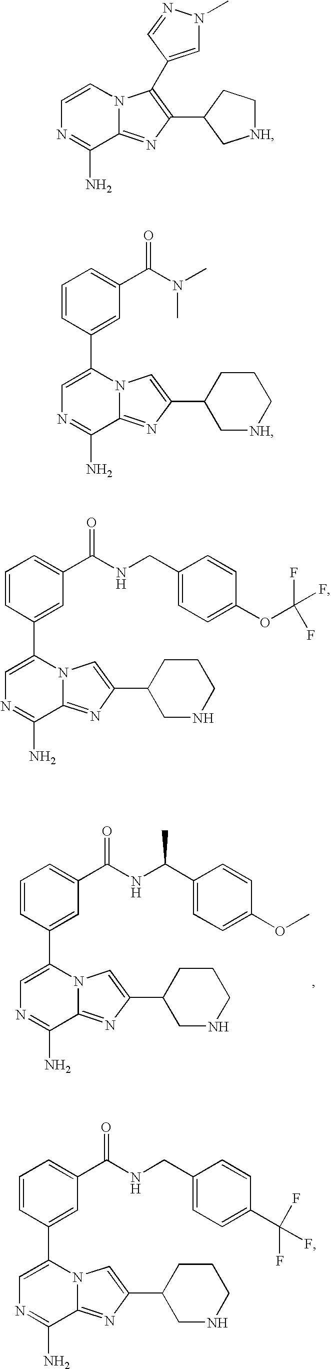 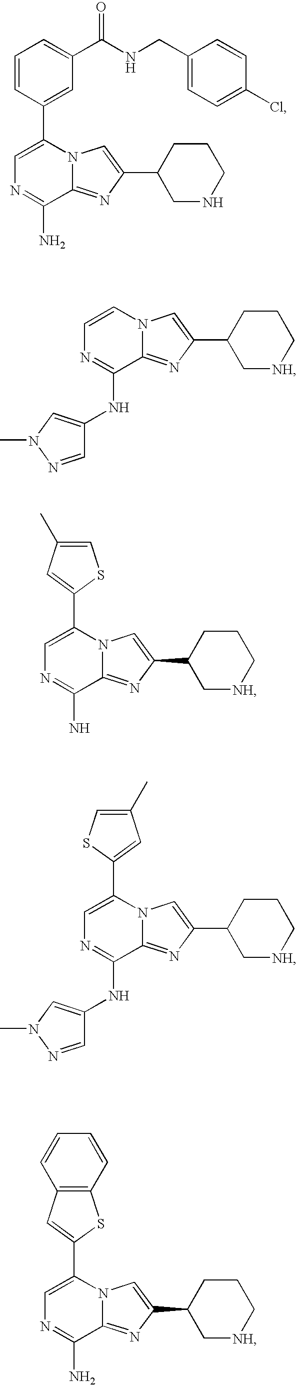 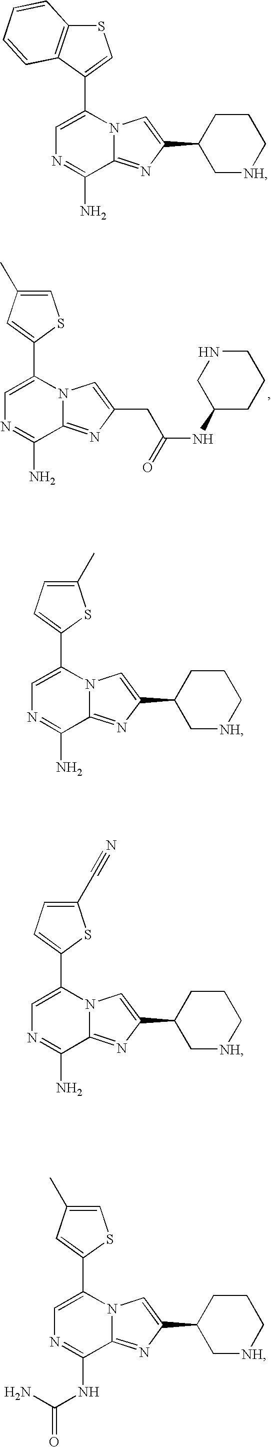 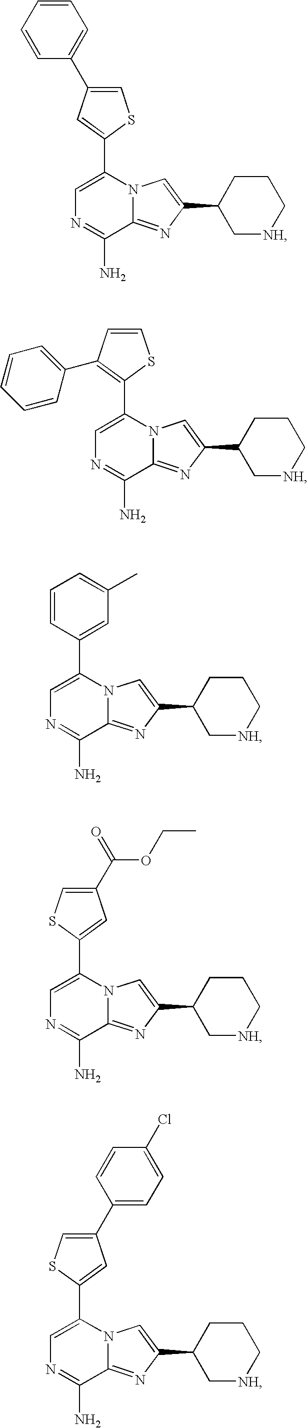 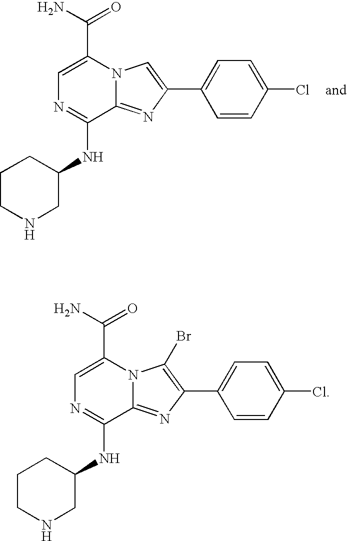
, or a pharmaceutically acceptable salt, solvate, ester or prodrug thereof; and
an amount of at least one second compound, the second compound being an anti-inflammatory agent different from the first compound;
wherein the amounts of the first compound and the second compound result in a therapeutic effect.

76. The method according to claim 75, wherein the anti-inflammatory agent is selected from the group consisting of disease modifying antirheumatic drugs NSAIDs, COX-2 inhibitors, COX-1 inhibitors, immunosuppressives, BRMs, anti-inflammatory agents and H1 antagonists disease modifying anti-rheumatic drugs (DMARDS), non-steroidal anti-inflammatory drugs (NSAIDs), cycloxygenase-2 selective (COX-2) inhibitors, COX-1 inhibitors, immunosuppressives, biological response modifiers (BRMs), anti-inflammatory agents and H1 antagonists.
